SEQUENCE LISTING (89 sequences)

SEQ ID NO: 1 - protein, 735 aa, Adenovirus2 [sequence omitted]

SEQ ID NO: 2 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSAPPRHPSEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRTTQFHPPEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRPKQPTQPKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRPSYSPSNQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSSVVSSRCEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRKNKDTPVKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRNQNETKRQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSKQLPTNNKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 10 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRQNPKLGSEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSKINPNASKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSTRNPARNQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRSMGRGLGEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRRQRIPGGEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSNHHTNNPKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRNYGRQDSQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSLPRRDAPKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSLDKKNATKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 19 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSSQRLPTTQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRPTKHLDREAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 21 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRQQPQNTRQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSTLQRTMAKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRHLAVAPPQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRPWRESSQEAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 25 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQSTTTRDMPKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRRKAENQMKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 27 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRRGSDPVRKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 28 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRKKNEETKKAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - protein, 742 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGQRRINMATGQAAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 30 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtgcgccgcctcggcatccgtctgaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 31 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggactacgcagtttcatccgccggaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 32 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggcctaagcagcctacgcagcctaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 33 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggccttcttatagtccttcgaatcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 34 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtagtgttgtgagttcgcgttgtgaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 35 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggaagaataaggatacgccggtgaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 36 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggaatcagaatgagactaagaggcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 37 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtaagcagcttccgactaataataaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 38 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggcagaatccgaagctgggttcggaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 39 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtaagattaatccgaatgcttcgaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 40 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtactcgtaatccggctaggaatcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 41 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggagtatggggcggggtctgggtgaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 42 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggcggcagaggattccggggggggaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 43 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtaatcatcatacgaataatccgaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 44 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggaattatggtaggcaggattctcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 45 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtttgcctcggcgggatgctcctaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 46 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtctggataagaagaatgcgacgaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 47 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtagtcagaggctgccgacgactcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 48 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggccgacgaagcatttggatcgggaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 49 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggcagcagcctcagaatactcgtcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 50 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtacgcttcagcggacgatggcgaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 51 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggcatctggctgtggcgccgccgcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 52 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggccttggcgtgagtcgtctcaggaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 53 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagagtacgactacgcgtgatatgcctaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 54 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggaggaaggcggagaatcagatgaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 55 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggagggggagtgatccggttaggaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 56 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggaagaagaatgaggagacgaagaaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 57 - DNA, 2229 bp, Adenovirus2
atggctgccgatggttatcttccagattggctcgaggacactctctctgaaggcattcgccagtggtggaagttgaaacctggaccaccgccacccaaacccgcagagcggcataaggacgacagccggggtcttgtgcttcctggttacaagtacctcggacccttcaacggactcgacaagggggagcccgtcaacgaggcggacgcagcggccctcgagcacgacaaggcctacgaccggcagctcgacagcggtgacaacccgtacctcaagtacaaccacgccgacgccgagtttcaggagcgtctgaaagaagatacgtcttttgggggcaacctcgggcgagcagtcttccaggccaagaagagggttctcgaacctcttggtctggttgaggaacctgttaagacggctcctggaaagaagcgtccggtagagcactctccagtggagccagactcctcctcgggcaccggcaaggcgggccagcagcccgctagaaagagactcaattttggtcagactggcgacgcagactcagtccccgaccctcaacctctcggacagcctccagcagccccctctggtttgggaactaatacaatggctacaggcagtggcgcaccaatggcagacaataacgaaggcgccgacggagtgggtaattcctcgggaaattggcattgcgattccacatggatgggcgacagagtcatcaccaccagcacccgaacctgggccctgcccacctacaacaaccacctctacaagcaaatcagtagccaatcaggagccagcaacgacaaccactacttcggctacagcaccccctgggggtattttgacttcaacagattccactgccacttctcaccacgtgactggcagcgactcatcaacaacaactggggattccggcccaagagactcaacttcaagctcttcaacatccaagtcaaggaggtcacgcagaatgatggcacgacgaccatcgccaataaccttaccagcacggttcaagtgtttacggactcggagtaccagctcccgtacgtcctcggctctgcgcaccagggctgcctccctccgttcccggcggacgtcttcatggtgccgcagtacggctacctgacgctgaacaacggcagccaggccgtgggacgctcatccttttactgcctggaatacttcccttcgcagatgctgagaacgggcaacaactttaccttcagctacacctttgaggacgtgcctttccacagcagctacgctcacagccagagcctggaccggctgatgaatcctctcatcgaccagtacctgtactacctgagcagaactaacactccatccggaaccaccacgcaatcaaggttgcagtttagccaggccggagcgagtgacattcgggaccagtctagaaactggctacctggaccctgttaccggcagcagcgcgtttcaaagacatctgcggacaacaacaacagcgaatactcgtggactggtgctaccaaatatcatctcaatggccgtgactctctggtgaatcctggcccggctatggcctcacacaaagacgacgaagaaaagttctttccccagagcggtgtcctcatttttggcaaacaaggctcagagaaaacaaacgtggatattgaaaaggtcatgattaccgacgaagaggaaattaggaccactaaccctgtggccacggagcagtatgggtctgtgtctaccaacctccagggccagaggcggattaatatggcgacggggcaggcggccgcgaccgcagatgtcaacacacagggagttttacctggcatggtgtggcaagacagagacgtgtacctgcagggtcccatttgggccaagattcctcacacggatggacactttcacccgtctccgctgatgggcggctttggactgaaacacccgcctcctcagatcctcatcaaaaacacgcctgtacctgcgaatccttcgaccaccttcagcgctgcaaagtttgcttcattcatcacccagtactccactggacaggtcagcgtggagattgagtgggagctgcagaaggaaaacagcaagcgctggaatcccgagatccagtacacatccaactacaacaaatctgttaatgtggactttactgtggacactaatggagtgtatagtgagcctcgccccattggcacccgttacctcacccgtaatctgtaa

SEQ ID NO: 58 - protein, 9 aa, Artificial Sequence
  Synthetic Sequence
SAPPRHPSE

SEQ ID NO: 59 - protein, 9 aa, Artificial Sequence
  Synthetic Sequence
RTTQFHPPE

SEQ ID NO: 60 - protein, 9 aa, Artificial Sequence
  Synthetic Sequence
RPKQPTQPK

SEQ ID NO: 61 - protein, 9 aa, Artificial Sequence
  Synthetic Sequence
RPSYSPSNQ

SEQ ID NO: 62 - protein, 9 aa, Artificial Sequence
  Synthetic Sequence
SSVVSSRCE

SEQ ID NO: 63 - protein, 9 aa, Artificial Sequence
  Synthetic Sequence
RKNKDTPVK

SEQ ID NO: 64 - protein, 9 aa, Artificial Sequence
  Synthetic Sequence
RNQNETKRQ

SEQ ID NO: 65 - protein, 9 aa, Artificial Sequence
  Synthetic Sequence
SKQLPTNNK

SEQ ID NO: 66 - protein, 9 aa, Artificial Sequence
  Synthetic Sequence
RQNPKLGSE

SEQ ID NO: 67 - protein, 9 aa, Artificial Sequence
  Synthetic Sequence
SKINPNASK

SEQ ID NO: 68 - protein, 9 aa, Artificial Sequence
  Synthetic Sequence
STRNPARNQ

SEQ ID NO: 69 - protein, 9 aa, Artificial Sequence
  Synthetic Sequence
RSMGRGLGE

SEQ ID NO: 70 - protein, 9 aa, Artificial Sequence
  Synthetic Sequence
RRQRIPGGE

SEQ ID NO: 71 - protein, 9 aa, Artificial Sequence
  Synthetic Sequence
SNHHTNNPK

SEQ ID NO: 72 - protein, 9 aa, Artificial Sequence
  Synthetic Sequence
RNYGRQDSQ

SEQ ID NO: 73 - protein, 9 aa, Artificial Sequence
  Synthetic Sequence
SLPRRDAPK

SEQ ID NO: 74 - protein, 9 aa, Artificial Sequence
  Synthetic Sequence
SLDKKNATK

SEQ ID NO: 75 - protein, 9 aa, Artificial Sequence
  Synthetic Sequence
SSQRLPTTQ

SEQ ID NO: 76 - protein, 9 aa, Artificial Sequence
  Synthetic Sequence
RPTKHLDRE

SEQ ID NO: 77 - protein, 9 aa, Artificial Sequence
  Synthetic Sequence
RQQPQNTRQ

SEQ ID NO: 78 - protein, 9 aa, Artificial Sequence
  Synthetic Sequence
STLQRTMAK

SEQ ID NO: 79 - protein, 9 aa, Artificial Sequence
  Synthetic Sequence
RHLAVAPPQ

SEQ ID NO: 80 - protein, 9 aa, Artificial Sequence
  Synthetic Sequence
RPWRESSQE

SEQ ID NO: 81 - protein, 9 aa, Artificial Sequence
  Synthetic Sequence
STTTRDMPK

SEQ ID NO: 82 - protein, 9 aa, Artificial Sequence
  Synthetic Sequence
RRKAENQMK

SEQ ID NO: 83 - protein, 9 aa, Artificial Sequence
  Synthetic Sequence
RRGSDPVRK

SEQ ID NO: 84 - protein, 9 aa, Artificial Sequence
  Synthetic Sequence
RKKNEETKK

SEQ ID NO: 85 - protein, 9 aa, Artificial Sequence
  Synthetic Sequence
RRINMATGQ

SEQ ID NO: 86 - protein, 745 aa, Adenovirus2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 87 - protein, 738 aa, Adenovirus2
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 88 - protein, 733 aa, Adenovirus2
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 89 - protein, 736 aa, Adenovirus2
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

(19) United States
(12) Patent Application Publication
(10) Pub. No.: US 2024/0059743 A1
(43) Pub. Date: Feb. 22, 2024

(54) AAV CAPSIDS AND VECTORS

(71) Applicants: Chidren's Medical Research Institute, Westmead (AU); The Sydney Children's Hospitals Network (Randwick and Westmead) (Incorporating The Royal Alexandra, Westmead (AU)

(72) Inventors: Adrian Westhaus, Westmead (AU); Lezek Lisowski, Westmead (AU); Robyn Jamieson, Westmead (AU); Steven Eamegdool, Parramatta (AU); Anai Gonzalez Cordero, North Sydney (AU)

(21) Appl. No.: 18/267,373

(22) PCT Filed: Dec. 16, 2021
(86) PCT No.: PCT/AU2021/051496

§ 371 Date: Jun. 14, 2023

(30) Foreign Application Priority Data
Dec. 16, 2020 (AU) 2020904689

(51) Int. Cl. C07K 14/075

(52) U.S. Cl. CPC C07K 14/075; C12N 2750/14122

(57) ABSTRACT

The present disclosure relates generally to adeno-associated vims (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

This application claims priority to Australian Provisional Application No. 2020904689 entitled “AAV capsids and vectors” filed 16 Dec. 2020, the content of which is incorporated herein by reference in its entirety.

### FIELD OF THE DISCLOSURE

The present disclosure relates generally to adeno-associated virus (AAV) capsid polypeptides and encoding nucleic acid molecules. The disclosure also relates to AAV vectors comprising the capsid polypeptides, and nucleic acid vectors (e.g. plasmids) comprising the encoding nucleic acids molecules, as well as to host cells comprising the vectors. The disclosure also relates to methods and uses of the polypeptides, encoding nucleic acids molecules, vectors and host cells.

### BACKGROUND OF THE DISCLOSURE

Gene therapy has most commonly been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) from an alternative open reading frame. AAP promotes capsid assembly, acting to target the capsid proteins to the nucleolus and promote capsid formation. The rep gene encodes four regulatory proteins: Rep78, Rep68, Rep52 and Rep40. These Rep proteins are involved in AAV genome replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV virus in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV, rAAV) that contain a genome that lacks some, most or all of the native AAV genome and instead contains one or more heterologous sequences flanked by the ITRs have been successfully used in gene therapy settings. These AAV vectors are widely used to deliver heterologous nucleic acid to cells of a subject for therapeutic purposes. Diseases and conditions of the central nervous system, including diseases and conditions of the retina or brain, are candidates for gene therapy using AAV. However, there are a limited number of AAV vectors that are efficient at transducing cells of the central nervous system (CNS), such as cells in the retina or brain, so as to treat, for example, inherited retinal diseases and other diseases associated with the CNS. There remains a need, therefore, to develop novel AAV vectors suitable for gene therapy of the CNS, and to provide methods for identifying and/or developing such AAV vectors.

### SUMMARY OF THE DISCLOSURE

The present disclosure is predicated in part on the identification of novel AAV capsid polypeptides. Typically, the capsid polypeptides, when present in the capsid of an AAV vector, facilitate transduction of cells, such as transduction of cells in the CNS, including cells in the retina and/or the brain. In some examples, the capsid polypeptides, when present in the capsid of an AAV vector, facilitate transduction of cells in the retina (such as, but not limited to, Müller glia cells, retinal pigment epithelium, photoreceptor cells, horizontal cells, bipolar cells, amacrine cells and/or ganglion retinal cells). The transduction of cells (e.g. cells of the human CNS) by AAV vectors having a capsid comprising a capsid polypeptide of the present disclosure is generally increased or enhanced compared to AAV vectors comprising a reference AAV capsid polypeptide (e.g. the prototypic AAV2 capsid set forth in SEQ ID NO:1). The capsid polypeptides of the present disclosure are therefore particularly useful in preparing AAV vectors, and in particular, AAV vectors for therapeutic applications. Similarly, AAV vectors comprising a capsid polypeptide of the present disclosure (i.e. having a capsid comprising or consisting of a capsid polypeptide of the present disclosure) are of particular use in gene therapy applications, such as for delivery of heterologous nucleic acids to the CNS (including the retina and/or the brain) for the treatment of various diseases and conditions.

In one aspect, provided is an AAV capsid polypeptide, comprising a peptide modification relative to the AAV2 capsid polypeptide set forth in SEQ ID NO:1, wherein: the peptide modification is in variable region 8 (VRVIII); the peptide modification comprises a 7 amino insertion relative to the AAV2 capsid polypeptide set forth in SEQ ID NO:1, and comprises the sequence set forth in any one of SEQ ID Nos:58-85; and the portion of the capsid polypeptide that is not the peptide modification comprises at least or about 80%, 81%, 82%, 83%, 84%, 85%, 86%, 87%, 88%, 89%, 90%, 91%, 92%, 93%, 94%, 95%, or 96% sequence identity to positions 1-735, 138-735 or 203-735 of SEQ ID NO:1.

In some examples, the peptide modification is in the region of the capsid polypeptide spanning positions 585-589, with numbering relative to SEQ ID NO:1. In one embodiment, the peptide modification comprises a 7 amino insertion after position 587 relative to the AAV2 capsid polypeptide set forth in SEQ ID NO:1. In particular embodiments, the peptide modification comprises amino acid substitutions at positions 587 and 588 relative to the AAV2 capsid polypeptide set forth in SEQ ID NO:1. In some examples, the AAV capsid polypeptide comprises one or more amino acid substitutions at position 585, 586, and/or 589 relative to the AAV2 capsid polypeptide set forth in SEQ ID NO:1 (e.g. R585G, G586Q and/or Q589A, relative to the AAV2 capsid polypeptide set forth in SEQ ID NO:1). In particular examples, the AAV capsid polypeptide comprises the sequence of amino acids set forth in any one of SEQ ID NOs:2-29, the sequence of amino acids set forth as amino acids 138-742 of any one of SEQ ID NOs:2-29, or the sequence of amino acids set forth as amino acids 203-742 of any one of SEQ ID NOs:2-29; or a sequence having at least or about 85%, 86%, 87%, 88%, 89%, 90%, 91%, 92%, 93%, 94%, 95%, 96%, 97%, 98%, or 99% sequence identity thereto. In exemplary embodiments, the peptide modification comprises the sequence of amino acids set forth in any one of SEQ ID NOs: 60, 62, 63, 64, 67, 70 and 73.

Also provided is an AAV capsid polypeptide, comprising: a) a VP1 protein comprising the sequence of amino acids set forth in any one of SEQ ID NOs:2-29; b) a VP2 protein comprising the sequence of amino acids set forth as amino acids 138-742 of any one of SEQ ID NOs:2-29; c) a VP3 protein comprising the sequence of amino acids set forth as amino acids 203-742 of any one of SEQ ID NOs:2-29; or d) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a)-c), wherein the capsid polypeptide comprises, at positions 587-595 with numbering relative to any one of SEQ ID NOs:2-29, the peptide sequence set forth in any one of SEQ ID Nos:58-85. In particular examples, the capsid polypeptide comprises, at positions 587-595 with numbering relative to any one of SEQ ID NOs:2-29, the peptide sequence set forth in any one of SEQ ID NOs: 60, 62, 63, 64, 67, 70 and 73.

In a further aspect, provided is an AAV vector, comprising a capsid polypeptide described herein. In some examples, the vector exhibits increased transduction efficiency of a cell of the CNS compared to an AAV vector comprising a capsid polypeptide comprising the sequence of amino acids set forth in SEQ ID NO: (e.g. wherein transduction efficiency is increased by at least or about 10%, 20%, 30%, 40%, 50%, 60%, 70%, 80%, 90%, 100%, 200%, 300%, 400% or 500%). In one example, the cell of the CNS is selected from among a Müller glia cell, retinal pigment epithelium cell, photoreceptor cell (rods and cones), horizontal cell bipolar cell, amacrine cell, retinal ganglion cell, neuron, astrocyte, oligodendrocyte, and microglia. In particular embodiments, the AAV vector further comprises a heterologous coding sequence, such as one that encodes a peptide, polypeptide or polynucleotide (e.g. a therapeutic peptide, polypeptide or polynucleotide).

Additional aspects relate to an isolated nucleic acid molecule encoding a capsid polypeptide described herein, and a vector (e.g. a plasmid, cosmid, phage and transposon) comprising the nucleic acid molecule. In another aspect, provided is a a host cell comprising a AAV vector, a nucleic acid molecule, or a vector described herein.

In a further aspect, provided is a method for introducing a heterologous coding sequence into a host cell, comprising contacting a host cell with an AAV vector described herein that comprises a heterologous coding sequence. The method can be in vitro, in vivo or ex vivo. In some examples, the host cell is a cell of the CNS. In one embodiment, contacting a host cell with the AAV vector comprises administering the AAV vector to a subject. In such embodiments, administration of the AAV vector to the subject may effect treatment of a disease or condition associated with the CNS.

In another aspect, provided is a method for producing an AAV vector, comprising culturing a host cell comprising a nucleic acid molecule encoding a capsid polypeptide described herein, an AAV rep gene, a heterologous coding sequence flanked by AAV inverted terminal repeats, and helper functions for generating a productive AAV infection, under conditions suitable to facilitate assembly of an AAV vector comprising a capsid comprising the capsid polypeptide, wherein the capsid encapsidates the heterologous coding sequence. In some examples, the host cell is a cell of the CNS (e.g. a Müller glia cell, retinal pigment epithelium cell, photoreceptor cell (rods and cones), horizontal cell bipolar cell, amacrine cell, retinal ganglion cell, neuron, astrocyte, oligodendrocyte, or microglia).

A further aspect of the disclosure relates to the use of a AAV vector described herein for the preparation of a medicament for treating a disease or condition associated with the CNS.

### BRIEF DESCRIPTION OF THE DRAWINGS

Embodiments of the disclosure are described herein, by way of non-limiting example only, with reference to the following drawings.

FIG. 1 is a schematic of the peptide library construction. Detailed view of the modification workflow of the Ico2 capsid. Amino acid position of Q584 and A591 using numbering from un-modified cap2 VP1. First step was the insertion of two SfiI restriction sites. Second step was the insertion of the peptide library. Seven truly randomized NNK (X1-X7) insertion as well as the full 9-mer peptide including semi-random flanking amino acids are shown within the modified region.

FIG. 2 is a schematic showing the selection system utilised for selection of new capsids. (A) A schematic of the functional transduction (FT) selection of the FT-SFFV-GFP.AAVLib^{2-PEP} library. The cap library was cloned into a construct under the control of the p40 promoter. The construct also contained the eGFP gene under the control of the SFFV promoter, and ITRs flanking the expression cassette. The plasmid was used to produce the AAV2-based FT-SFFV-GFP.AAVLib^{2-PEP} library, which was transduced into a floating human retinal explant for the first round of selection. DNA and RNA were extracted from the explants, processed for NGS analysis of selection progression and the RNA-derived peptide region was used to produce a secondary library for a further round of selection. (B) A schematic of the second round of selection with the enriched library from the RNA recovered from the first round of selection. Interphase retinal explants were transduced at three different doses (low, mid, high) with the secondary library, dissociated and RNA was extracted from unsorted cells of the low dose as well as CD73+ sorted cells of the mid and high dose. Top 5 candidates from the first round of selection and the three conditions of the second round of selection (n=20) were vectorized and further validated compared to 5 benchmark AAVs creating a ‘25 Retina AAV Kit’.

FIG. 3 is a schematic of the retinal explant culture systems used in the screening processes of FIG. 2. Retinal tissue dissected from donated human eye globes were used for establishment of a floating human retinal explant culture system (i). An interphase retinal explant culture system was established from another doner eye for the second round of selection (ii).

FIG. 4 shows the measured titres (vg/mL) of AAV variants in the 25 Retina AAV Kit, before dilution to 4×10¹² vg/mL (pre-dilution titre) and after dilution (dilution titre), and expressed as % of total reads in the diluted mix.

FIG. 5 provides a schematic and results of a study in which the ability of AAV vectors in the 25 Retina AAV Kit was assessed. (A) Schematic of the study in which the 25 Retina AAV Kit was used to transduce primary interphase retinal explants, iPSC-derived retina organoids and iPSC-derived (i) and primary (p) human cultured retina pigment epithelium (RPE) before DNA and RNA was obtained and processed for NGS. (B) Data from dissociated explants that were unsorted, CD73⁺ or CD73⁻ sorted before NGS was then performed from processed DNA and RNA. Data represent % of total NGS read for each row/population. (C) Data from iPSC-derived retinal organoids. NGS was performed from processed DNA and RNA. Data represent % of total NGS read for each row/population. (D) Data from iPSC-derived and primary human cultured RPE. NGS was performed from processed DNA and RNA. Data represent % of total NGS read for each row/population.

FIG. 6 shows the results of two studies assession the performance of vectors in organoids. (A) Performance of 25 selected capsids in iPSC-derived cortical and whole brain organoids. NGS was performed from processed DNA and RNA. Data represent % of total NGS read for each row/population. (B) shows data from performance of the 25 selected capsids in iPSC-derived whole brain organoids when harvested 4 or 14 days after transduction. NGS was performed from processed DNA and RNA. Data represent % of total NGS read for each row/population.

FIG. 7 is a photographic representation of AAV variant expression in retinal tissue through representative immunohistochemical staining of human retinal explants post-AAV transduction. GFAP indicates astrocytes and cells in the ganglion cell layer (GCL), to assist in orientation of the tissue. Photoreceptor layer (PL) is indicated at the bottom of the tissue. (A) Control; (B) AAV2-L1; (C) AAV2-L2; (D) AAV2-M1; (E) AAV2-M4; (F) AAV2-1.3. All capsids illustrated here show expression in the retina, and different capsids appear predominantly expressed in some cell types more than others. Scale bar=100 μm.

FIG. 8 shows the results of flow cytometry analysis of retinal organoids transduced with either AAV 7m8 or AAV2-M4. The human synapsinl promoter was coupled, in reverse orientation, to the AAV-p40 promoter driving GFP (hSYN-p40). Experiments were performed in three independently differentiated iPSC-derived retinal organoids.

FIG. 9 is a photographic representation of AAV variant expression and GFP expression in retinal tissue through immunohistochemical staining of retinal organoids transduced with either AAV 7m8 or AAV2-M4 using the SFFV promoter or human synapsinl promoter coupled, in reverse orientation, to p40, driving GFP expression (SFFVrv-p40 and hSYNrv-p40, respectively). Scale bar=20 μm.

FIG. 10 shows transgene expression (at cDNA level) in brain, cervical spine and lumbar spine tissue from an adult mouse injected intrathecally with a kit comprising AAV2, AAV8, AAV9, AAV 7m8, AAV2-M1 and AAV2-L5 capsids. Error bars are from two separate BCs. For brain, different regions were processed and graphed together for simplicity.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication deficient. Exemplary polynucleotide vectors include, but are not limited to, plasmids, cosmids and transposons. Exemplary viral vectors include, for example, AAV, lentivira I, retrovira I, adenoviral, herpes viral and hepatitis viral vectors.

As used herein, “adeno-associated viral vector” or “AAV vector” refers to a vector in which the capsid is derived from an adeno-associated virus, including without limitation, AAV1, AAV2, AAV3, AAV4, AAV5, AAV6, AAV7, AAV8, AAV9, AAV10, AAV11, AAV12 or AAV13, AAV from other clades or isolates, or is derived from synthetic, bioengineered or modified AAV capsid proteins, including chimeric capsid proteins. In particular embodiments, the AAV vector has a capsid comprising a capsid polypeptide of the present disclosure. When referring to AAV vectors, both the source of the genome and the source of the capsid can be identified, where the source of the genome is the first number designated and the source of the capsid is the second number designated. Thus, for example, a vector in which both the capsid and genome are derived from AAV2 is more accurately referred to as AAV2/2. A vector with an AAV6-derived capsid and an AAV2-derived genome is most accurately referred to as AAV2/6. A vector with the bioengineered DJ capsid and an AAV2-derived genome is most accurately referred to as AAV2/DJ. For simplicity, and because most vectors use an AAV2-derived genome, it is understood that reference to an AAV6 vector generally refers to an AAV2/6 vector, reference to an AAV2 vector generally refers to an AAV2/2 vector, etc. An AAV vector may also be referred to herein as “recombinant AAV”, “rAAV”, “recombinant AAV virion”, “rAAV virion”, “AAV variant”, “recombinant AAV variant”, and “rAAV variant” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, “functional” with reference to a capsid polypeptide means that the polypeptide can self-assemble or assemble with different capsid polypeptides to produce the proteinaceous shell (capsid) of an AAV virion. It is to be understood that not all capsid polypeptides in a given host cell assemble into AAV capsids. Preferably, at least 25%, at least 50%, at least 75%, at least 85%, at least 90%, at least 95% of all AAV capsid polypeptide molecules assemble into AAV capsids. Suitable assays for measuring this biological activity are described e.g. in Smith-Arica and Bartlett (2001), Curr Cardiol Rep 3(1): 43-49.

“AAV helper functions” or “helper functions” refer to functions that allow AAV to be replicated and packaged by a host cell. AAV helper functions can be provided in any of a number of forms, including, but not limited to, as a helper virus or as helper virus genes which aid in AAV replication and packaging. Helper virus genes include, but are not limited to, adenoviral helper genes such as E1A, E1B, E2A, E4 and VA. Helper viruses include, but are not limited to, adenoviruses, herpesviruses, poxviruses such as vaccinia, and baculovirus. The adenoviruses encompass a number of different subgroups, although Adenovirus type 5 of subgroup C (Ad5) is most commonly used. Numerous adenoviruses of human, non-human mammalian and avian origin are known and are available from depositories such as the ATCC. Viruses of the herpes family, which are also available from depositories such as ATCC, include, for example, herpes simplex viruses (HSV), Epstein-Barr viruses (EBV), cytomegaloviruses (CMV) and pseudorabies viruses (PRV). Baculoviruses available from depositories include Autographa californica nuclear polyhedrosis virus.

As used herein, the term “transduction” refers to the ability of an AAV vector to enter one or more particular cell types and transfer the DNA contained within the AAV vector into the cell. Transduction can be assessed by measuring the amount of AAV DNA or RNA expressed from the AAV DNA in a cell or population of cells, and/or by assessing the number of cells in a population that contain AAV DNA or RNA expressed from the DNA. Where the presence or amount of RNA is assessed, the type of transduction assessed is referred to herein as “functional transduction”, i.e. the ability of the AAV to transfer DNA to the cell and have that DNA expressed. “Transduction efficiency” is a measure of the level of transduction from a starting amount of AAV vector (e.g. the starting amount of vector being injected in vivo or applied to cells in vitro), and can be quantitative or qualitative, and/or with reference to a particular control, e.g. a prototypic AAV vector. For example, if a candidate AAV vector transduces twice as many cells as a control vector and/or the amount of AAV DNA per cell from transduction with the candidate AAV vector is twice that of transduction with the control vector, where the starting amount of each vector was the same (i.e. the amount of each vector injected into a subject or applied to cells was the same), it can be said that the transduction efficiency of the candidate AAV vector is 200% greater than, or is twice that of, the transduction efficiency of the control vector.

The phrase “numbering relative to” a sequence, such as SEQ ID NO:1, means that the numbering of the amino acid position being referred to is as shown in the sequence, e.g. SEQ ID NO:1. It will be appreciated that the sequence is simply a reference sequence, and that the same amino acid residue or position may correspond to a different number in a different sequence, such as if the different sequence is a truncated form or is a sequence that has insertions or deletions compared to the reference sequence. To identify corresponding positions or residues in different sequences, sequences of related or variant polypeptides are aligned by any method known to those of skill in the art. Such methods typically maximize matches (e.g. identical nucleotides or amino acids at positions), and include methods such as using manual alignments and by using the numerous alignment programs available (for example, BLASTP, ClustIW, ClustIW2, EMBOSS, LALIGN, Kalign, etc.) and others known to those of skill in the art. By aligning the sequences of polypeptides, one skilled in the art can identify corresponding positions. For example, by aligning the prototypic AAV2 capsid polypeptide set forth in SEQ ID NO:1 with another AAV capsid polypeptide, such as the AAV2-RC01 capsid set forth in SEQ ID NO:2, one of skill in the art can identify regions or amino acids residues within AAV2-RC01 that correspond to various regions or residues in the AAV2 polypeptide set forth in SEQ ID NO:1. For example, the leucine at position 735 of SEQ ID NO:1 corresponds to the leucine at position 742 of SEQ ID NO:2.

As used herein, “corresponding nucleotides” or “corresponding amino acid residues” or grammatical variations thereof refer to nucleotides or amino acids that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustIW, ClustIW2, EMBOSS, LALIGN, Kalign, etc) and others known to those of skill in the art. By aligning the sequences of polynucleotides or polypeptides, one skilled in the art can identify corresponding nucleotides or amino acids. For example, by aligning the prototypic AAV2 capsid polypeptide set forth in SEQ ID NO:1 with another AAV capsid polypeptide, such as the variant set forth in SEQ ID NO:2, one of skill in the art can identify regions or amino acids residues within the other AAV polypeptide that correspond to various regions or residues in the AAV polypeptide set forth in SEQ ID NO:1, e.g. the asparagine at position 734 of SEQ ID NO:1 corresponds to the asparagine at position 741 of SEQ ID NO:2.

The term “peptide modification” refers to a modification in a polypeptide that involves two or more contiguous amino acids (i.e. that involves a peptide within the polypeptide). The peptide modification can include amino acid insertions, deletions and/or substitutions relative to a reference polypeptide. For example, an exemplary peptide modification of the present disclosure comprises 9 consecutive amino acid residues, wherein 7 of those residues are insertions relative to the prototypic AAV2 capsid set forth in SEQ ID NO:1, and 2 of those residues are amino acid substitutions relative to the prototypic AAV2 capsid set forth in SEQ ID NO:1.

A “heterologous coding sequence” as used herein refers to nucleic acid sequence present in a polynucleotide, vector, or host cell that is not naturally found in the polynucleotide, vector, or host cell or is not naturally found at the position that it is at in the polynucleotide, vector, or host cell, i.e. is non-native. A “heterologous coding sequence” can encode a peptide or polypeptide, or a polynucleotide that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous sequence and the genomic DNA can occur. In one example, the heterologous coding sequence is a functional copy of a gene for introduction into a cell that has a defective/mutated copy.

As used herein, the term “operably-linked” with reference to a promoter and a coding sequence means that the transcription of the coding sequence is under the control of, or driven by, the promoter.

The term “host cell” refers to a cell, such as a mammalian cell, that has introduced into it the exogenous DNA, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, “isolated” with reference to a polynucleotide or polypeptide means that the polynucleotide or polypeptide is substantially free of cellular material or other contaminating proteins from the cells from which the polynucleotide or polypeptide is derived, or substantially free from chemical precursors or other chemicals when chemically synthesized.

The term “subject” as used herein refers to an animal, in particular a mammal and more particularly a primate including a lower primate and even more particularly, a human who can benefit from the present invention. A subject, regardless of whether a human or non-human animal or embryo, may be referred to as an individual, subject, animal, patient, host or recipient. The present disclosure has both human and veterinary applications. For convenience, an “animal” specifically includes livestock animals such as cattle, horses, sheep, pigs, camelids, goats and donkeys, as well as domestic animals, such as dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates. In some embodiments, the subject is human.

It will be appreciated that the above described terms and associated definitions are used for the purpose of explanation only and are not intended to be limiting.

TABLE 1
Description of Sequences

| SEQ | |
|---|---|
| ID | |
| NO | Sequence description |
| 1 | AAV2 prototypic capsid (VP1 protein) |
| 2 | AAV2-1.1 capsid (VP1 protein) |
| 3 | AAV2-1.2 capsid (VP1 protein) |
| 4 | AAV2-1.3 capsid (VP1 protein) |
| 5 | AAV2-1.4 capsid (VP1 protein) |
| 6 | AAV2-1.5 capsid (VP1 protein) |
| 7 | AAV2-L1 capsid (VP1 protein) |
| 8 | AAV2-L2 capsid (VP1 protein) |
| 9 | AAV2-L3 capsid (VP1 protein) |
| 10 | AAV2-L4 capsid (VP1 protein) |
| 11 | AAV2-L5 capsid (VP1 protein) |
| 12 | AAV2-L6 capsid (VP1 protein) |
| 13 | AAV2-L7 capsid (VP1 protein) |
| 14 | AAV2-M1 capsid (VP1 protein) |
| 15 | AAV2-M2 capsid (VP1 protein) |
| 16 | AAV2-M3 capsid (VP1 protein) |
| 17 | AAV2-M4 capsid (VP1 protein) |
| 18 | AAV2-M5 capsid (VP1 protein) |
| 19 | AAV2-M6 capsid (VP1 protein) |
| 20 | AAV2-M7 capsid (VP1 protein) |
| 21 | AAV2-H1 capsid (VP1 protein) |
| 22 | AAV2-H2 capsid (VP1 protein) |
| 23 | AAV2-H3 capsid (VP1 protein) |
| 24 | AAV2-H4 capsid (VP1 protein) |
| 25 | AAV2-H5 capsid (VP1 protein) |
| 26 | AAV2-H6 capsid (VP1 protein) |
| 27 | AAV2-H7 capsid (VP1 protein) |
| 28 | AAV2-H9 capsid (VP1 protein) |
| 29 | AAV2-H11 capsid (VP1 protein) |
| 30 | AAV2-1.1 capsid (VP1 nucleic acid) |
| 31 | AAV2-1.2 capsid (VP1 nucleic acid) |
| 32 | AAV2-1.3 capsid (VP1 nucleic acid) |
| 33 | AAV2-1.4 capsid (VP1 nucleic acid) |
| 34 | AAV2-1.5 capsid (VP1 nucleic acid) |
| 35 | AAV2-L1 capsid (VP1 nucleic acid) |
| 36 | AAV2-L2 capsid (VP1 nucleic acid) |
| 37 | AAV2-L3 capsid (VP1 nucleic acid) |
| 38 | AAV2-L4 capsid (VP1 nucleic acid) |
| 39 | AAV2-L5 capsid (VP1 nucleic acid) |
| 40 | AAV2-L6 capsid (VP1 nucleic acid) |
| 41 | AAV2-L7 capsid (VP1 nucleic acid) |
| 42 | AAV2-M1 capsid (VP1 nucleic acid) |
| 43 | AAV2-M2 capsid (VP1 nucleic acid) |
| 44 | AAV2-M3 capsid (VP1 nucleic acid) |
| 45 | AAV2-M4 capsid (VP1 nucleic acid) |
| 46 | AAV2-M5 capsid (VP1 nucleic acid) |
| 47 | AAV2-M6 capsid (VP1 nucleic acid) |
| 48 | AAV2-M7 capsid (VP1 nucleic acid) |
| 49 | AAV2-H1 capsid (VP1 nucleic acid) |
| 50 | AAV2-H2 capsid (VP1 nucleic acid) |
| 51 | AAV2-H3 capsid (VP1 nucleic acid) |
| 52 | AAV2-H4 capsid (VP1 nucleic acid) |
| 53 | AAV2-H5 capsid (VP1 nucleic acid) |
| 54 | AAV2-H6 capsid (VP1 nucleic acid) |
| 55 | AAV2-H7 capsid (VP1 nucleic acid) |
| 56 | AAV2-H9 capsid (VP1 nucleic acid) |
| 57 | AAV2-H11 capsid (VP1 nucleic acid) |
| 58 | SAPPRHPSE peptide modification sequence |
| | (AAV2-1.1 capsid) |
| 59 | RTTQFHPPE peptide modification sequence |
| | (AAV2-1.2 capsid) |
| 60 | RPKQPTQPK peptide modification sequence |
| | (AAV2-1.3 capsid) |
| 61 | RPSYSPSNQ peptide modification sequence |
| | (AAV2-1.4 capsid) |
| 62 | SSVVSSRCE peptide modification sequence |
| | (AAV2-1.5 capsid) |
| 63 | RKNKDTPVK peptide modification sequence |
| | (AAV2-L1 capsid) |
| 64 | RNQNETKRQ peptide modification sequence |
| | (AAV2-L2 capsid) |
| 65 | SKQLPTNNK peptide modification sequence |
| | (AAV2-L3 capsid) |
| 66 | RQNPKLGSE peptide modification sequence |
| | (AAV2-L4 capsid) |
| 67 | SKINPNASK peptide modification sequence |
| | (AAV2-L5 capsid) |
| 68 | STRNPARNQ peptide modification sequence |
| | (AAV2-L6 capsid) |
| 69 | RSMGRGLGE peptide modification sequence |
| | (AAV2-L7 capsid) |
| 70 | RRQRIPGGE peptide modification sequence |
| | (AAV2-M1 capsid) |
| 71 | SNHHTNNPK peptide modification sequence |
| | (AAV2-M2 capsid) |
| 72 | RNYGRQDSQ peptide modification sequence |
| | (AAV2-M3 capsid) |
| 73 | SLPRRDAPK peptide modification sequence |
| | (AAV2-M4 capsid) |
| 74 | SLDKKNATK peptide modification sequence |
| | (AAV2-M5 capsid) |
| 75 | SSQRLPTTQ peptide modification sequence |
| | (AAV2-M6 capsid) |
| 76 | RPTKHLDRE peptide modification sequence |
| | (AAV2-M7 capsid) |
| 77 | RQQPQNTRQ peptide modification sequence |
| | (AAV2-H1 capsid) |
| 78 | STLQRTMAK peptide modification sequence |
| | (AAV2-H2 capsid) |
| 79 | RHLAVAPPQ peptide modification sequence |
| | (AAV2-H3 capsid) |
| 80 | RPWRESSQE peptide modification sequence |
| | (AAV2-H4 capsid) |
| 81 | STTTRDMPK peptide modification sequence |
| | (AAV2-H5 capsid) |
| 82 | RRKAENQMK peptide modification sequence |
| | (AAV2-H6 capsid) |
| 83 | RRGSDPVRK peptide modification sequence |
| | (AAV2-H7 capsid) |
| 84 | RKKNEETKK peptide modification sequence |
| | (AAV2-H8 capsid) |
| 85 | RRINMATGQ peptide modification sequence |
| | (AAV2-H9 capsid) |
| 86 | AAV 7m8 capsid (VP1 protein) |
| 87 | AAV8 capsid (VP1 protein) |
| 88 | AAV13 capsid (VP1 protein) |
| 89 | AAV Anc80L65 (VP1 protein) |

### Capsid Polypeptides

The present disclosure is predicated in part on the identification of novel AAV capsid polypeptides. Typically, the capsid polypeptides, when present in the capsid of an AAV vector, facilitate transduction of cells, such as cells in the retina or brain or of other cells in the CNS. In a particular aspect, the present disclosure relates to AAV capsid polypeptides that facilitate transduction of cells in the retina, such as, but not limited to, Müller glia cells, retinal pigment epithelium, photoreceptor cells (rods and cones), horizontal cells, bipolar cells, amacrine cells and/or retinal ganglion cells). In other aspects, the present disclosure relates to AAV capsid polypeptides that facilitate transduction of cells in the brain (e.g. a neuron, oligodendrocyte, astrocyte or microglia). The transduction of cells (e.g. human retinal cells, brain cells or other cells of the CNS) by AAV vectors having a capsid comprising a capsid polypeptide of the present disclosure is generally increased or enhanced compared to AAV vectors comprising a reference AAV capsid polypeptide (e.g. the prototypic AAV2 capsid set forth in SEQ ID NO:1). Transduction or transduction efficiency of AAV vectors can be increased by at least or about 10%, 20%, 30%, 40%, 50%, 60%, 70%, 80%, 90%, 100%, 200%, 300%, 400%, 500%, 600%, 700%, 800%, 900%, 1000% or more, e.g. an AAV vector comprising a capsid polypeptide of the present disclosure can be at least or about 1.2×, 1.5×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more efficient at transducing cells in vivo compared to an AAV vector comprising a reference AAV capsid polypeptide (e.g. one set forth in SEQ ID NO:1). In particular examples, the increased transduction or transduction efficiency of the AAV vector is observed in cells of the human retina, such as, for example, in Müller glia cells, retinal pigment epithelium and/or photoreceptor cells. In other examples, the increased transduction or transduction efficiency of the AAV vector is observed in cells of the human brain.

The capsid polypeptides of the present disclosure are therefore particularly useful in preparing AAV vectors, such as AAV vectors for therapy of retinal diseases or conditions, diseases or conditions of the brain, and/or other diseases or conditions of ther CNS, whereby delivery of heterologous nucleic acid into the cell by the AAV vector facilitaes therapy.

The AAV capsid polypeptides of the present disclosure (including isolated capsid polypeptides) include those having a peptide modification in variable region 8 (VRVIII) relative to a reference AAV capsid polypeptide, such as the prototypic AAV2 capsid polypeptide set forth in SEQ ID NO:1 (where VRVIII spans amino acids 579-594 of SEQ ID NO:1). The peptide modification comprises a 7 amino acid insertion relative to the AAV2 capsid polypeptide set forth in SEQ ID NO:1, wherein the 7 amino acid insertion comprises a sequence set forth at amino acids 2-8 of any one of SEQ ID NOs:58-85. Typically, the peptide modification comprises 9 consecutive amino acid residues having a sequence set forth in any one of SEQ ID NOs: 58-85, which includes the 7 amino acid insertion. The peptide modification can be at any location in VRVIII. In one example, the peptide modification is in the region spanning positions 585-589, with numbering relative to SEQ ID NO:1. In one example, the peptide modification comprises the 7 amino acid insertion after the amino acid residue at positions 587 relative to SEQ ID NO:1, and optionally amino acid substitutions at positions 587 and 588 relative to the AAV2 capsid polypeptide set forth in SEQ ID NO:1, e.g. substitution of the amino acid at position 587 with an arginine or serine, and/or substitution of the amino acid at position 588 with an glutamic acid, glutamine or lysine. In further embodiments, there is, or is also, one or more amino acid substitutions at position 585, 586, and/or 589 relative to SEQ ID NO:1, such as R585G, G586Q and/or Q589A relative to SEQ ID NO:1. The capsid polypeptides of the present disclosure can include all or a portion of the VP1 protein (comprising amino acid residues corresponding to those at positions 1-735 of SEQ ID NO:1), VP2 protein (comprising amino acid residues corresponding to those at positions 138-735 of SEQ ID NO:1) and/or the VP3 protein (comprising amino acid residues corresponding to those at positions 203-735 of SEQ ID NO:1). The capsid polypeptides typically comprise at least or about 80%, 81%, 82%, 83%, 84%, 85%, 86%, 87%, 88%, 89%, 90%, 91%, 92%, 93%, 94%, 95%, 96%, 97%, 98% or 99% sequence identity to the VP1, VP2 or VP3 proteins of the prototypic AAV2 set forth in SEQ ID NO:1. In particular embodiments, the portion of the capsid polypeptide that is not the peptide modification comprises at least or about 80%, 81%, 82%, 83%, 84%, 85%, 86%, 87%, 88%, 89%, 90%, 91%, 92%, 93%, 94%, 95%, or 96% sequence identity to the VP1, VP2 or VP3 proteins of the prototypic AAV2 set forth in SEQ ID NO:1.

Thus, provided herein are polypeptides, including isolated polypeptides, comprising all or a portion of an AAV capsid polypeptide set forth in any one of SEQ ID NOs: 2-29, including all or a portion of the VP1 protein (comprising amino acid residues corresponding to those at positions 1-735 of SEQ ID NO:1), VP2 protein (comprising amino acid residues corresponding to those at positions 138-735 of SEQ ID NO:1) and/or the VP3 protein (comprising amino acid residues corresponding to those at positions 203-735 of SEQ ID NO:1), and variants thereof, including variants comprising at least or about 85%, 86%, 87%, 88%, 89%, 90%, 91%, 92%, 93%, 94%, 95%, 96%, 97%, 98%, or 99% sequence identity to the VP1, VP2 or VP3 proteins described herein. Capsid polypeptides of the disclosure therefore include those comprising all or a portion of the VP1 protein set forth in any one of SEQ ID NOs:2-29 or a polypeptide having at least or about 85%, 86%, 87%, 88%, 89%, 90%, 91%, 92%, 93%, 94%, 95%, 96%, 97%, 98%, or 99% sequence identity thereto, wherein the polypeptide comprises a 9 amino acid sequence set forth in any one of SEQ ID NOs: 58-85 at positions 587-595 relative to SEQ ID NOs:2-29, or a 7 amino acid sequence set forth as amino acids 2-8 of any one of SEQ ID NOs: 58-85 at positions 588-594 relative to SEQ ID NOs:2-29. Also included in the present disclosure are capsid polypeptides comprising all or a portion of the VP2 protein set forth as amino acids 138-742 of any one of SEQ ID NOs:2-29 or comprising a sequence having at least or about 85%, 86%, 87%, 88%, 89%, 90%, 91%, 92%, 93%, 94%, 95%, 96%, 97%, 98%, or 99% sequence identity to the VP2 protein set forth as amino acids 138-742 of SEQ ID NOs:2-29 or a functional fragment thereof, wherein the polypeptide comprises a 9 amino acid sequence set forth in any one of SEQ ID NOs: 58-85 at positions 587-595 relative to SEQ ID NOs:2-29, or a 7 amino acid sequence set forth as amino acids 2-8 of any one of SEQ ID NOs: 58-85 at positions 588-594 relative to SEQ ID NOs:2-29. In addition, provided are capsid polypeptides comprising all or a portion of the VP3 protein set forth as amino acids 203-742 of any one of SEQ ID NOs:2-29 or comprising a sequence having at least or about 85%, 86%, 87%, 88%, 89%, 90%, 91%, 92%, 93%, 94%, 95%, 96%, 97%, 98%, or 99% sequence identity to the VP3 protein set forth as amino acids 203-742 of any one of SEQ ID NOs:2-29 or a functional fragment thereof, wherein the polypeptide comprises a 9 amino acid sequence set forth in any one of SEQ ID NOs: 58-85 at positions 587-595 relative to SEQ ID NOs:2-29, or a 7 amino acid sequence set forth as amino acids 2-8 of any one of SEQ ID NOs: 58-85 at positions 588-594 relative to SEQ ID NOs:2-29.

An exemplary capsid polypeptide, AAV2-1.1 (SEQ ID NO:2), comprises a peptide modification in VRVIII relative to the prototypic AAV2 set forth in SEQ ID NO:1, wherein the peptide modification comprises the sequence SAPPRHPSE (SEQ ID NO:58), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SAPPRHPSE (SEQ ID NO:58) or the sequence set forth at positions 2-8 of SEQ ID NO:58, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:2, the VP3 protein set forth as amino acids 203-742 of SEQ ID NO:2, or the VP2 protein set forth as amino acids 138-742 of SEQ ID NO:2 (e.g. the capsid polypeptide comprises a) the sequence of the VP1 protein set forth in SEQ ID NO:2, the VP3 protein set forth as amino acids 203-742 of SEQ ID NO:2, or the VP2 protein set forth as amino acids 138-742 of SEQ ID NO:2; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SAPPRHPSE (SEQ ID NO:58) at positions 587-595 or the sequence set forth at amino acid positions 2-8 of SEQ ID NO:59 at positions 588-594, with numbering relative to SEQ ID NO:2). In particular embodiments, the portion of the capsid polypeptide that is not the peptide modification (e.g. that is not the 9 amino acid residues set forth in SEQ ID NO:58 at positions 587-595, with numbering relative to SEQ ID NO:2) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-1.2 (SEQ ID NO:3) comprises a peptide modification in VRVIII relative to the prototypic AAV2 set forth in SEQ ID NO:1, wherein the peptide modification comprises the sequence RTTQFHPPE (SEQ ID NO:59), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RTTQFHPPE (SEQ ID NO:59) or the sequence set forth at positions 2-8 of SEQ ID NO:59, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:3, the VP3 protein set forth as amino acids 203-742 of SEQ ID NO:3, or the VP2 protein set forth as amino acids 138-742 of SEQ ID NO:3 (e.g. the capsid polypeptide comprises a) the sequence of the VP1 protein set forth in SEQ ID NO:3, the VP3 protein set forth as amino acids 203-742 of SEQ ID NO:2, or the VP2 protein set forth as amino acids 138-742 of SEQ ID NO:3; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RTTQFHPPE (SEQ ID NO:59) at positions 587-595 or the sequence set forth at amino acid positions 2-8 of SEQ ID NO:59 at positions 588-594, with numbering relative to SEQ ID NO:3). In particular embodiments, the portion of the capsid polypeptide that is not the peptide modification (e.g. that is not the 9 amino acid residues set forth in SEQ ID NO:59 at positions 587-595, with numbering relative to SEQ ID NO:3) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-1.3 (SEQ ID NO:4) comprises a peptide modification in VRVIII relative to the prototypic AAV2 set forth in SEQ ID NO:1, wherein the peptide modification comprises the sequence RPKQPTQPK (SEQ ID NO:60), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RPKQPTQPK (SEQ ID NO:60) or the sequence set forth at positions 2-8 of SEQ ID NO:60, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:4, the VP3 protein set forth as amino acids 203-742 of SEQ ID NO:4, or the VP2 protein set forth as amino acids 138-742 of SEQ ID NO:4 (e.g. the capsid polypeptide comprises a) the sequence of the VP1 protein set forth in SEQ ID NO:4, the VP3 protein set forth as amino acids 203-742 of SEQ ID NO:2, or the VP2 protein set forth as amino acids 138-742 of SEQ ID NO:4; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RPKQPTQPK (SEQ ID NO:60) at positions 587-595 or the sequence set forth at amino acid positions 2-8 of SEQ ID NO:60 at positions 588-594, with numbering relative to SEQ ID NO:4). In particular embodiments, the portion of the capsid polypeptide that is not the peptide modification (e.g. that is not the 9 amino acid residues set forth in SEQ ID NO:60 at positions 587-595, with numbering relative to SEQ ID NO:4) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-1.4 (SEQ ID NO:5) comprises a peptide modification in VRVIII relative to the prototypic AAV2 set forth in SEQ ID NO:1, wherein the peptide modification comprises the sequence RPSYSPSNQ (SEQ ID NO:61), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RPSYSPSNQ (SEQ ID NO:61) or the sequence set forth at positions 2-8 of SEQ ID NO:61, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:5, the VP3 protein set forth as amino acids 203-742 of SEQ ID NO:5, or the VP2 protein set forth as amino acids 138-742 of SEQ ID NO:5 (e.g. the capsid polypeptide comprises a) the sequence of the VP1 protein set forth in SEQ ID NO:5, the VP3 protein set forth as amino acids 203-742 of SEQ ID NO:5, or the VP2 protein set forth as amino acids 138-742 of SEQ ID NO:5; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RPSYSPSNQ (SEQ ID NO:61) at positions 587-595 or the sequence set forth at amino acid positions 2-8 of SEQ ID NO:61 at positions 588-594, with numbering relative to SEQ ID NO:5). In particular embodiments, the portion of the capsid polypeptide that is not the peptide modification (e.g. that is not the 9 amino acid residues set forth in SEQ ID NO:61 at positions 587-595, with numbering relative to SEQ ID NO:5) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-1.5 (SEQ ID NO:6) comprises a peptide modification in VRVIII relative to the prototypic AAV2 set forth in SEQ ID NO:1, wherein the peptide modification comprises the sequence SSVVSSRCE (SEQ ID NO:62), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SSVVSSRCE (SEQ ID NO:62) or the sequence set forth at positions 2-8 of SEQ ID NO:62, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:6, the VP3 protein set forth as amino acids 203-742 of SEQ ID NO:6, or the VP2 protein set forth as amino acids 138-742 of SEQ ID NO:6 (e.g. the capsid polypeptide comprises a) the sequence of the VP1 protein set forth in SEQ ID NO:6, the VP3 protein set forth as amino acids 203-742 of SEQ ID NO:6, or the VP2 protein set forth as amino acids 138-742 of SEQ ID NO:6; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SSVVSSRCE (SEQ ID NO:62) at positions 587-595 or the sequence set forth at amino acid positions 2-8 of SEQ ID NO:62 at positions 588-594, with numbering relative to SEQ ID NO:6). In particular embodiments, the portion of the capsid polypeptide that is not the peptide modification (e.g. that is not the 9 amino acid residues set forth in SEQ ID NO:62 at positions 587-595, with numbering relative to SEQ ID NO:6) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-L1(SEQ ID NO:7) comprises a peptide modification in VRVIII relative to the prototypic AAV2 set forth in SEQ ID NO:1, wherein the peptide modification comprises the sequence RKNKDTPVK (SEQ ID NO:63), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RKNKDTPVK (SEQ ID NO:63) or the sequence set forth at positions 2-8 of SEQ ID NO:63, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:7, the VP3 protein set forth as amino acids 203-742 of SEQ ID NO:7, or the VP2 protein set forth as amino acids 138-742 of SEQ ID NO:7 (e.g. the capsid polypeptide comprises a) the sequence of the VP1 protein set forth in SEQ ID NO:7, the VP3 protein set forth as amino acids 203-742 of SEQ ID NO:7, or the VP2 protein set forth as amino acids 138-742 of SEQ ID NO:7; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RKNKDTPVK (SEQ ID NO:63) at positions 587-595 or the sequence set forth at amino acid positions 2-8 of SEQ ID NO:63 at positions 588-594, with numbering relative to SEQ ID NO:7). In particular embodiments, the portion of the capsid polypeptide that is not the peptide modification (e.g. that is not the 9 amino acid residues set forth in SEQ ID NO:63 at positions 587-595, with numbering relative to SEQ ID NO:7) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-L2 (SEQ ID NO:8) comprises a peptide modification in VRVIII relative to the prototypic AAV2 set forth in SEQ ID NO:1, wherein the peptide modification comprises the sequence RNQNETKRQ (SEQ ID NO:64), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RNQNETKRQ (SEQ ID NO:64) or the sequence set forth at positions 2-8 of SEQ ID NO:64, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:8, the VP3 protein set forth as amino acids 203-742 of SEQ ID NO:8, or the VP2 protein set forth as amino acids 138-742 of SEQ ID NO:8 (e.g. the capsid polypeptide comprises a) the sequence of the VP1 protein set forth in SEQ ID NO:8, the VP3 protein set forth as amino acids 203-742 of SEQ ID NO:8, or the VP2 protein set forth as amino acids 138-742 of SEQ ID NO:8; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RNQNETKRQ (SEQ ID NO:64) at positions 587-595 or the sequence set forth at amino acid positions 2-8 of SEQ ID NO:64 at positions 588-594, with numbering relative to SEQ ID NO:8). In particular embodiments, the portion of the capsid polypeptide that is not the peptide modification (e.g. that is not the 9 amino acid residues set forth in SEQ ID NO:64 at positions 587-595, with numbering relative to SEQ ID NO:8) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-L3 (SEQ ID NO:9) comprises a peptide modification in VRVIII relative to the prototypic AAV2 set forth in SEQ ID NO:1, wherein the peptide modification comprises the sequence SKQLPTNNK (SEQ ID NO:65), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SKQLPTNNK (SEQ ID NO:65) or the sequence set forth at positions 2-8 of SEQ ID NO:65, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:9, the VP3 protein set forth as amino acids 203-742 of SEQ ID NO:9, or the VP2 protein set forth as amino acids 138-742 of SEQ ID NO:9 (e.g. the capsid polypeptide comprises a) the sequence of the VP1 protein set forth in SEQ ID NO:9, the VP3 protein set forth as amino acids 203-742 of SEQ ID NO:9, or the VP2 protein set forth as amino acids 138-742 of SEQ ID NO:9; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SKQLPTNNK (SEQ ID NO:65) at positions 587-595 or the sequence set forth at amino acid positions 2-8 of SEQ ID NO:65 at positions 588-594, with numbering relative to SEQ ID NO:9). In particular embodiments, the portion of the capsid polypeptide that is not the peptide modification (e.g. that is not the 9 amino acid residues set forth in SEQ ID NO:65 at positions 587-595, with numbering relative to SEQ ID NO:9) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-L4 (SEQ ID NO:10) comprises a peptide modification in VRVIII relative to the prototypic AAV2 set forth in SEQ ID NO:1, wherein the peptide modification comprises the sequence RQNPKLGSE (SEQ ID NO:66), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RQNPKLGSE (SEQ ID NO:66) or the sequence set forth at positions 2-8 of SEQ ID NO:66, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:10, the VP3 protein set forth as amino acids 203-742 of SEQ ID NO:10, or the VP2 protein set forth as amino acids 138-742 of SEQ ID NO:10 (e.g. the capsid polypeptide comprises a) the sequence of the VP1 protein set forth in SEQ ID NO:10, the VP3 protein set forth as amino acids 203-742 of SEQ ID NO:10, or the VP2 protein set forth as amino acids 138-742 of SEQ ID NO:10; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RQNPKLGSE (SEQ ID NO:66) at positions 587-595 or the sequence set forth at amino acid positions 2-8 of SEQ ID NO:66 at positions 588-594, with numbering relative to SEQ ID NO:10). In particular embodiments, the portion of the capsid polypeptide that is not the peptide modification (e.g. that is not the 9 amino acid residues set forth in SEQ ID NO:66 at positions 587-595, with numbering relative to SEQ ID NO:10) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-L5 (SEQ ID NO:11) comprises a peptide modification in VRVIII relative to the prototypic AAV2 set forth in SEQ ID NO:1, wherein the peptide modification comprises the sequence SKINPNASK (SEQ ID NO:67), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SKINPNASK (SEQ ID NO:67) or the sequence set forth at positions 2-8 of SEQ ID NO:67, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:11, the VP3 protein set forth as amino acids 203-742 of SEQ ID NO:11, or the VP2 protein set forth as amino acids 138-742 of SEQ ID NO:11 (e.g. the capsid polypeptide comprises a) the sequence of the VP1 protein set forth in SEQ ID NO:11, the VP3 protein set forth as amino acids 203-742 of SEQ ID NO:11, or the VP2 protein set forth as amino acids 138-742 of SEQ ID NO:11; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SKINPNASK (SEQ ID NO:67) at positions 587-595 or the sequence set forth at amino acid positions 2-8 of SEQ ID NO:67 at positions 588-594, with numbering relative to SEQ ID NO:11). In particular embodiments, the portion of the capsid polypeptide that is not the peptide modification (e.g. that is not the 9 amino acid residues set forth in SEQ ID NO:67 at positions 587-595, with numbering relative to SEQ ID NO:11) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-L6 (SEQ ID NO:12) comprises a peptide modification in VRVIII relative to the prototypic AAV2 set forth in SEQ ID NO:1, wherein the peptide modification comprises the sequence STRNPARNQ (SEQ ID NO:68), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence STRNPARNQ (SEQ ID NO:68) or the sequence set forth at positions 2-8 of SEQ ID NO:68, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:12, the VP3 protein set forth as amino acids 203-742 of SEQ ID NO:12, or the VP2 protein set forth as amino acids 138-742 of SEQ ID NO:12 (e.g. the capsid polypeptide comprises a) the sequence of the VP1 protein set forth in SEQ ID NO:12, the VP3 protein set forth as amino acids 203-742 of SEQ ID NO:12, or the VP2 protein set forth as amino acids 138-742 of SEQ ID NO:12; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence STRNPARNQ (SEQ ID NO:68) at positions 587-595 or the sequence set forth at amino acid positions 2-8 of SEQ ID NO:68 at positions 588-594, with numbering relative to SEQ ID NO:12). In particular embodiments, the portion of the capsid polypeptide that is not the peptide modification (e.g. that is not the 9 amino acid residues set forth in SEQ ID NO:68 at positions 587-595, with numbering relative to SEQ ID NO:12) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-L7 (SEQ ID NO:13) comprises a peptide modification in VRVIII relative to the prototypic AAV2 set forth in SEQ ID NO:1, wherein the peptide modification comprises the sequence RSMGRGLGE (SEQ ID NO:69), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RSMGRGLGE (SEQ ID NO:69) or the sequence set forth at positions 2-8 of SEQ ID NO:69, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:13, the VP3 protein set forth as amino acids 203-742 of SEQ ID NO:13, or the VP2 protein set forth as amino acids 138-742 of SEQ ID NO:13 (e.g. the capsid polypeptide comprises a) the sequence of the VP1 protein set forth in SEQ ID NO:13, the VP3 protein set forth as amino acids 203-742 of SEQ ID NO:13, or the VP2 protein set forth as amino acids 138-742 of SEQ ID NO:13; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RSMGRGLGE (SEQ ID NO:69) at positions 587-595 or the sequence set forth at amino acid positions 2-8 of SEQ ID NO:69 at positions 588-594, with numbering relative to SEQ ID NO:13). In particular embodiments, the portion of the capsid polypeptide that is not the peptide modification (e.g. that is not the 9 amino acid residues set forth in SEQ ID NO:69 at positions 587-595, with numbering relative to SEQ ID NO:13) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-M1 (SEQ ID NO:14) comprises a peptide modification in VRVIII relative to the prototypic AAV2 set forth in SEQ ID NO:1, wherein the peptide modification comprises the sequence RRQRIPGGE (SEQ ID NO:70), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RRQRIPGGE (SEQ ID NO:70) or the sequence set forth at positions 2-8 of SEQ ID NO:70, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:14, the VP3 protein set forth as amino acids 203-742 of SEQ ID NO:14, or the VP2 protein set forth as amino acids 138-742 of SEQ ID NO:14 (e.g. the capsid polypeptide comprises a) the sequence of the VP1 protein set forth in SEQ ID NO:14, the VP3 protein set forth as amino acids 203-742 of SEQ ID NO:14, or the VP2 protein set forth as amino acids 138-742 of SEQ ID NO:14; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RRQRIPGGE (SEQ ID NO:70) at positions 587-595 or the sequence set forth at amino acid positions 2-8 of SEQ ID NO:70 at positions 588-594, with numbering relative to SEQ ID NO:14). In particular embodiments, the portion of the capsid polypeptide that is not the peptide modification (e.g. that is not the 9 amino acid residues set forth in SEQ ID NO:70 at positions 587-595, with numbering relative to SEQ ID NO:14) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide AAV2-M2 (SEQ ID NO:15) comprises a peptide modification in VRVIII relative to the prototypic AAV2 set forth in SEQ ID NO:1, wherein the peptide modification comprises the sequence SNHHTNNPK (SEQ ID NO:71), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SNHHTNNPK (SEQ ID NO:71) or the sequence set forth at positions 2-8 of SEQ ID NO:71, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:15, the VP3 protein set forth as amino acids 203-742 of SEQ ID NO:15, or the VP2 protein set forth as amino acids 138-742 of SEQ ID NO:15 (e.g. the capsid polypeptide comprises a) the sequence of the VP1 protein set forth in SEQ ID NO:15, the VP3 protein set forth as amino acids 203-742 of SEQ ID NO:15, or the VP2 protein set forth as amino acids 138-742 of SEQ ID NO:15; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SNHHTNNPK (SEQ ID NO:71) at positions 587-595 or the sequence set forth at amino acid positions 2-8 of SEQ ID NO:71 at positions 588-594, with numbering relative to SEQ ID NO:15). In particular embodiments, the portion of the capsid polypeptide that is not the peptide modification (e.g. that is not the 9 amino acid residues set forth in SEQ ID NO:71 at positions 587-595, with numbering relative to SEQ ID NO:15) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide AAV2-M3 (SEQ ID NO:16) comprises a peptide modification in VRVIII relative to the prototypic AAV2 set forth in SEQ ID NO:1, wherein the peptide modification comprises the sequence RNYGRQDSQ (SEQ ID NO:72), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RNYGRQDSQ (SEQ ID NO:72) or the sequence set forth at positions 2-8 of SEQ ID NO:72, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:16, the VP3 protein set forth as amino acids 203-742 of SEQ ID NO:16, or the VP2 protein set forth as amino acids 138-742 of SEQ ID NO:16 (e.g. the capsid polypeptide comprises a) the sequence of the VP1 protein set forth in SEQ ID NO:16, the VP3 protein set forth as amino acids 203-742 of SEQ ID NO:16, or the VP2 protein set forth as amino acids 138-742 of SEQ ID NO:16; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RNYGRQDSQ (SEQ ID NO:72) at positions 587-595 or the sequence set forth at amino acid positions 2-8 of SEQ ID NO:72 at positions 588-594, with numbering relative to SEQ ID NO:16). In particular embodiments, the portion of the capsid polypeptide that is not the peptide modification (e.g. that is not the 9 amino acid residues set forth in SEQ ID NO:72 at positions 587-595, with numbering relative to SEQ ID NO:16) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide AAV2-M4 (SEQ ID NO:17) comprises a peptide modification in VRVIII relative to the prototypic AAV2 set forth in SEQ ID NO:1, wherein the peptide modification comprises the sequence SLPRRDAPK (SEQ ID NO:73), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SLPRRDAPK (SEQ ID NO:73) or the sequence set forth at positions 2-8 of SEQ ID NO:73, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:17, the VP3 protein set forth as amino acids 203-742 of SEQ ID NO:17, or the VP2 protein set forth as amino acids 138-742 of SEQ ID NO:17 (e.g. the capsid polypeptide comprises a) the sequence of the VP1 protein set forth in SEQ ID NO:17, the VP3 protein set forth as amino acids 203-742 of SEQ ID NO:17, or the VP2 protein set forth as amino acids 138-742 of SEQ ID NO:17; or b) a sequence having at least or about 85%, 86%, 87%, 88%, 89%, 90%, 91%, 92%, 93%, 94%, 95%, 96%, 97%, 98%, or 99% the sequence SLPRRDAPK (SEQ ID NO:73) at positions 587-595 or the sequence set forth at amino acid positions 2-8 of SEQ ID NO:73 at positions 588-594, with numbering relative to SEQ ID NO:17). In particular embodiments, the portion of the capsid polypeptide that is not the peptide modification (e.g. that is not the 9 amino acid residues set forth in SEQ ID NO:73 at positions 587-595, with numbering relative to SEQ ID NO:17) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide AAV2-M5 (SEQ ID NO:18) comprises a peptide modification in VRVIII relative to the prototypic AAV2 set forth in SEQ ID NO:1, wherein the peptide modification comprises the sequence SLDKKNATK (SEQ ID NO:74), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SLDKKNATK (SEQ ID NO:74) or the sequence set forth at positions 2-8 of SEQ ID NO:74, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:18, the VP3 protein set forth as amino acids 203-742 of SEQ ID NO:18, or the VP2 protein set forth as amino acids 138-742 of SEQ ID NO:18 (e.g. the capsid polypeptide comprises a) the sequence of the VP1 protein set forth in SEQ ID NO:18, the VP3 protein set forth as amino acids 203-742 of SEQ ID NO:18, or the VP2 protein set forth as amino acids 138-742 of SEQ ID NO:18; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SLDKKNATK (SEQ ID NO:74) at positions 587-595 or the sequence set forth at amino acid positions 2-8 of SEQ ID NO:74 at positions 588-594, with numbering relative to SEQ ID NO:18). In particular embodiments, the portion of the capsid polypeptide that is not the peptide modification (e.g. that is not the 9 amino acid residues set forth in SEQ ID NO:74 at positions 587-595, with numbering relative to SEQ ID NO:18) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide AAV2-M6 (SEQ ID NO:19) comprises a peptide modification in VRVIII relative to the prototypic AAV2 set forth in SEQ ID NO:1, wherein the peptide modification comprises the sequence SSQRLPTTQ (SEQ ID NO:75), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence SSQRLPTTQ (SEQ ID NO:75) or the sequence set forth at positions 2-8 of SEQ ID NO:75, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:19, the VP3 protein set forth as amino acids 203-742 of SEQ ID NO:19, or the VP2 protein set forth as amino acids 138-742 of SEQ ID NO:19 (e.g. the capsid polypeptide comprises a) the sequence of the VP1 protein set forth in SEQ ID NO:19, the VP3 protein set forth as amino acids 203-742 of SEQ ID NO:19, or the VP2 protein set forth as amino acids 138-742 of SEQ ID NO:19; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence SSVVSSRCE (SEQ ID NO:62) at positions 587-595 or the sequence set forth at amino acid positions 2-8 of SEQ ID NO:62 at positions 588-594, with numbering relative to SEQ ID NO:19). In particular embodiments, the portion of the capsid polypeptide that is not the peptide modification (e.g. that is not the 9 amino acid residues set forth in SEQ ID NO:62 at positions 587-595, with numbering relative to SEQ ID NO:19) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-M7 (SEQ ID NO:20) comprises a peptide modification in VRVIII relative to the prototypic AAV2 set forth in SEQ ID NO:1, wherein the peptide modification comprises the sequence RPTKHLDRE (SEQ ID NO:76), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RPTKHLDRE (SEQ ID NO:76) or the sequence set forth at positions 2-8 of SEQ ID NO:76, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:20, the VP3 protein set forth as amino acids 203-742 of SEQ ID NO:20, or the VP2 protein set forth as amino acids 138-742 of SEQ ID NO:20 (e.g. the capsid polypeptide comprises a) the sequence of the VP1 protein set forth in SEQ ID NO:20, the VP3 protein set forth as amino acids 203-742 of SEQ ID NO:20, or the VP2 protein set forth as amino acids 138-742 of SEQ ID NO:20; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RPTKHLDRE (SEQ ID NO:76) at positions 587-595 or the sequence set forth at amino acid positions 2-8 of SEQ ID NO:76 at positions 588-594, with numbering relative to SEQ ID NO:20). In particular embodiments, the portion of the capsid polypeptide that is not the peptide modification (e.g. that is not the 9 amino acid residues set forth in SEQ ID NO:76 at positions 587-595, with numbering relative to SEQ ID NO:20) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-H1 (SEQ ID NO:21) comprises a peptide modification in VRVIII relative to the prototypic AAV2 set forth in SEQ ID NO:1, wherein the peptide modification comprises the sequence RQQPQNTRQ (SEQ ID NO:77), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RQQPQNTRQ (SEQ ID NO:77) or the sequence set forth at positions 2-8 of SEQ ID NO:77, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:21, the VP3 protein set forth as amino acids 203-742 of SEQ ID NO:21, or the VP2 protein set forth as amino acids 138-742 of SEQ ID NO:21 (e.g. the capsid polypeptide comprises a) the sequence of the VP1 protein set forth in SEQ ID NO:21, the VP3 protein set forth as amino acids 203-742 of SEQ ID NO:21, or the VP2 protein set forth as amino acids 138-742 of SEQ ID NO:21; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RQQPQNTRQ (SEQ ID NO:77) at positions 587-595 or the sequence set forth at amino acid positions 2-8 of SEQ ID NO:77 at positions 588-594, with numbering relative to SEQ ID NO:21). In particular embodiments, the portion of the capsid polypeptide that is not the peptide modification (e.g. that is not the 9 amino acid residues set forth in SEQ ID NO:77 at positions 587-595, with numbering relative to SEQ ID NO:21) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-H2 (SEQ ID NO:22) comprises a peptide modification in VRVIII relative to the prototypic AAV2 set forth in SEQ ID NO:1, wherein the peptide modification comprises the sequence STLQRTMAK (SEQ ID NO:78), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence STLQRTMAK (SEQ ID NO:78) or the sequence set forth at positions 2-8 of SEQ ID NO:78, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:22, the VP3 protein set forth as amino acids 203-742 of SEQ ID NO:22, or the VP2 protein set forth as amino acids 138-742 of SEQ ID NO:22 (e.g. the capsid polypeptide comprises a) the sequence of the VP1 protein set forth in SEQ ID NO:22, the VP3 protein set forth as amino acids 203-742 of SEQ ID NO:22, or the VP2 protein set forth as amino acids 138-742 of SEQ ID NO:22; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence STLQRTMAK (SEQ ID NO:78) at positions 587-595 or the sequence set forth at amino acid positions 2-8 of SEQ ID NO:78 at positions 588-594, with numbering relative to SEQ ID NO:22). In particular embodiments, the portion of the capsid polypeptide that is not the peptide modification (e.g. that is not the 9 amino acid residues set forth in SEQ ID NO:78 at positions 587-595, with numbering relative to SEQ ID NO:22) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-H3 (SEQ ID NO:23) comprises a peptide modification in VRVIII relative to the prototypic AAV2 set forth in SEQ ID NO:1, wherein the peptide modification comprises the sequence RHLAVAPPQ (SEQ ID NO:79), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RHLAVAPPQ (SEQ ID NO:79) or the sequence set forth at positions 2-8 of SEQ ID NO:79, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:23, the VP3 protein set forth as amino acids 203-742 of SEQ ID NO:23, or the VP2 protein set forth as amino acids 138-742 of SEQ ID NO:23 (e.g. the capsid polypeptide comprises a) the sequence of the VP1 protein set forth in SEQ ID NO:23, the VP3 protein set forth as amino acids 203-742 of SEQ ID NO:23, or the VP2 protein set forth as amino acids 138-742 of SEQ ID NO:23; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RHLAVAPPQ (SEQ ID NO:79) at positions 587-595 or the sequence set forth at amino acid positions 2-8 of SEQ ID NO:79 at positions 588-594, with numbering relative to SEQ ID NO:23). In particular embodiments, the portion of the capsid polypeptide that is not the peptide modification (e.g. that is not the 9 amino acid residues set forth in SEQ ID NO:79 at positions 587-595, with numbering relative to SEQ ID NO:23) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-H4 (SEQ ID NO:24) comprises a peptide modification in VRVIII relative to the prototypic AAV2 set forth in SEQ ID NO:1, wherein the peptide modification comprises the sequence RPWRESSQE (SEQ ID NO:80), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RPWRESSQE (SEQ ID NO:80) or the sequence set forth at positions 2-8 of SEQ ID NO:80, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:24, the VP3 protein set forth as amino acids 203-742 of SEQ ID NO:24, or the VP2 protein set forth as amino acids 138-742 of SEQ ID NO:24 (e.g. the capsid polypeptide comprises a) the sequence of the VP1 protein set forth in SEQ ID NO:24, the VP3 protein set forth as amino acids 203-742 of SEQ ID NO:24, or the VP2 protein set forth as amino acids 138-742 of SEQ ID NO:24; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RPWRESSQE (SEQ ID NO:80) at positions 587-595 or the sequence set forth at amino acid positions 2-8 of SEQ ID NO:80 at positions 588-594, with numbering relative to SEQ ID NO:24). In particular embodiments, the portion of the capsid polypeptide that is not the peptide modification (e.g. that is not the 9 amino acid residues set forth in SEQ ID NO:80 at positions 587-595, with numbering relative to SEQ ID NO:24) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-H5 (SEQ ID NO:25) comprises a peptide modification in VRVIII relative to the prototypic AAV2 set forth in SEQ ID NO:1, wherein the peptide modification comprises the sequence STTTRDMPK (SEQ ID NO:81), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence STTTRDMPK (SEQ ID NO:81) or the sequence set forth at positions 2-8 of SEQ ID NO:81, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:25, the VP3 protein set forth as amino acids 203-742 of SEQ ID NO:25, or the VP2 protein set forth as amino acids 138-742 of SEQ ID NO:25 (e.g. the capsid polypeptide comprises a) the sequence of the VP1 protein set forth in SEQ ID NO:25, the VP3 protein set forth as amino acids 203-742 of SEQ ID NO:25, or the VP2 protein set forth as amino acids 138-742 of SEQ ID NO:25; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence STTTRDMPK (SEQ ID NO:81) at positions 587-595 or the sequence set forth at amino acid positions 2-8 of SEQ ID NO:81 at positions 588-594, with numbering relative to SEQ ID NO:25). In particular embodiments, the portion of the capsid polypeptide that is not the peptide modification (e.g. that is not the 9 amino acid residues set forth in SEQ ID NO:81 at positions 587-595, with numbering relative to SEQ ID NO:25) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-H6 (SEQ ID NO:26) comprises a peptide modification in VRVIII relative to the prototypic AAV2 set forth in SEQ ID NO:1, wherein the peptide modification comprises the sequence RRKAENQMK (SEQ ID NO:82), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RRKAENQMK (SEQ ID NO:82) or the sequence set forth at positions 2-8 of SEQ ID NO:82, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:26, the VP3 protein set forth as amino acids 203-742 of SEQ ID NO:26, or the VP2 protein set forth as amino acids 138-742 of SEQ ID NO:26 (e.g. the capsid polypeptide comprises a) the sequence of the VP1 protein set forth in SEQ ID NO:26, the VP3 protein set forth as amino acids 203-742 of SEQ ID NO:26, or the VP2 protein set forth as amino acids 138-742 of SEQ ID NO:26; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RRKAENQMK (SEQ ID NO:82) at positions 587-595 or the sequence set forth at amino acid positions 2-8 of SEQ ID NO:82 at positions 588-594, with numbering relative to SEQ ID NO:26). In particular embodiments, the portion of the capsid polypeptide that is not the peptide modification (e.g. that is not the 9 amino acid residues set forth in SEQ ID NO:82 at positions 587-595, with numbering relative to SEQ ID NO:26) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-H7 (SEQ ID NO:27) comprises a peptide modification in VRVIII relative to the prototypic AAV2 set forth in SEQ ID NO:1, wherein the peptide modification comprises the sequence RRGSDPVRK (SEQ ID NO:83), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RRGSDPVRK (SEQ ID NO:83) or the sequence set forth at positions 2-8 of SEQ ID NO:83, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:27, the VP3 protein set forth as amino acids 203-742 of SEQ ID NO:27, or the VP2 protein set forth as amino acids 138-742 of SEQ ID NO:27 (e.g. the capsid polypeptide comprises a) the sequence of the VP1 protein set forth in SEQ ID NO:27, the VP3 protein set forth as amino acids 203-742 of SEQ ID NO:27, or the VP2 protein set forth as amino acids 138-742 of SEQ ID NO:27; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RRGSDPVRK (SEQ ID NO:83) at positions 587-595 or the sequence set forth at amino acid positions 2-8 of SEQ ID NO:83 at positions 588-594, with numbering relative to SEQ ID NO:27). In particular embodiments, the portion of the capsid polypeptide that is not the peptide modification (e.g. that is not the 9 amino acid residues set forth in SEQ ID NO:83 at positions 587-595, with numbering relative to SEQ ID NO:27) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Another exemplary capsid polypeptide, AAV2-H9 (SEQ ID NO:28) comprises a peptide modification in VRVIII relative to the prototypic AAV2 set forth in SEQ ID NO:1, wherein the peptide modification comprises the sequence RKKNEETKK (SEQ ID NO:84), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RKKNEETKK (SEQ ID NO:84) or the sequence set forth at positions 2-8 of SEQ ID NO:84, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:28, the VP3 protein set forth as amino acids 203-742 of SEQ ID NO:28, or the VP2 protein set forth as amino acids 138-742 of SEQ ID NO:28 (e.g. the capsid polypeptide comprises a) the sequence of the VP1 protein set forth in SEQ ID NO:28, the VP3 protein set forth as amino acids 203-742 of SEQ ID NO:28, or the VP2 protein set forth as amino acids 138-742 of SEQ ID NO:28; or b) a sequence having at least or about 85%, 86%, 87%, 88%, 89%, 90%, 91%, 92%, 93%, 94%, 95%, 96%, 97%, 98%, or 99% sequence identity to the VP1, VP3 or VP2 proteins in a), wherein the capsid polypeptide comprises the sequence RKKNEETKK (SEQ ID NO:84) at positions 587-595 or the sequence set forth at amino acid positions 2-8 of SEQ ID NO:84 at positions 588-594, with numbering relative to SEQ ID NO:28). In particular embodiments, the portion of the capsid polypeptide that is not the peptide modification (e.g. that is not the 9 amino acid residues set forth in SEQ ID NO:84 at positions 587-595, with numbering relative to SEQ ID NO:28) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

A further exemplary capsid polypeptide, AAV2-H11 (SEQ ID NO:29) comprises a peptide modification in VRVIII relative to the prototypic AAV2 set forth in SEQ ID NO:1, wherein the peptide modification comprises the sequence RRINMATGQ (SEQ ID NO:85), which includes a 7 amino acid insertion after position 587 relative to the prototypic AAV2 capsid polypeptide set forth in SEQ ID NO:1 and amino acid substitutions at positions 587 and 588 relative to the AAV2 capsid polypeptide set forth in SEQ ID NO:1. Thus, in some embodiments, provided are capsid polypeptides comprising a peptide modification in VRVIII relative to SEQ ID NO:1, wherein the peptide modification comprises the sequence RRINMATGQ (SEQ ID NO:85) or the sequence set forth at positions 2-8 of SEQ ID NO:85, and wherein the capsid polypeptide has at least or about 85%, 86%, 87%, 88%, 89%, 90%, 91%, 92%, 93%, 94%, 95%, 96%, 97%, 98%, or 99% sequence identity to the VP1 protein set forth in SEQ ID NO:29, the VP3 protein set forth as amino acids 203-742 of SEQ ID NO:29, or the VP2 protein set forth as amino acids 138-742 of SEQ ID NO:29 (e.g. the capsid polypeptide comprises a) the sequence of the VP1 protein set forth in SEQ ID NO:29, the VP3 protein set forth as amino acids 203-742 of SEQ ID NO:29, or the VP2 protein set forth as amino acids 138-742 of SEQ ID NO:29; or b) a sequence having at least or about 85%, 86%, 87%, 88%, 89%, 90%, 91%, 92%, 93%, 94%, 95%, 96%, 97%, 98%, or 99% the sequence RRINMATGQ (SEQ ID NO:85) at positions 587-595 or the sequence set forth at amino acid positions 2-8 of SEQ ID NO:85 at positions 588-594, with numbering relative to SEQ ID NO:29). In particular embodiments, the portion of the capsid polypeptide that is not the peptide modification (e.g. that is not the 9 amino acid residues set forth in SEQ ID NO:85 at positions 587-595, with numbering relative to SEQ ID NO:29) has at least or about 85%, 86%, 87%, 88%, 89%, 90%, 91%, 92%, 93%, 94%, 95%, 96%, 97%, 98%, or 99% sequence identity to the VP1 protein, VP2 protein or VP3 protein set forth in SEQ ID NO:1.

Also provided are nucleic acid molecules, including isolated nucleic acid molecules, encoding a capsid polypeptide of the disclosure. Thus, amongst the nucleic acid molecules provided herein are those encoding a capsid polypeptide comprising the VP1, VP2 and/or VP3 of any one of the capsid polypeptides set forth in SEQ ID NOs:2-29 as described above or a polypeptides having at least or about 85%, 86%, 87%, 88%, 89%, 90%, 91%, 92%, 93%, 94%, 95%, 96%, 97%, 98%, or 99% sequence identity thereto. Non-limiting examples of nucleic acid molecules therefore include those set forth in SEQ ID NOs:30-57, those having at least or about 85%, 86%, 87%, 88%, 89%, 90%, 91%, 92%, 93%, 94%, 95%, 96%, 97%, 98%, or 99% sequence identity thereto, and those that hybridize with medium or high stringency to nucleic acid molecules comprising a sequence set forth in any one of SEQ ID NOs:30-57.

### Vectors

The present disclosure also provides vectors comprising a nucleic acid molecule that encodes a capsid polypeptide described herein, and vectors comprising a capsid polypeptide described herein. The vectors include nucleic acid vectors that comprise a nucleic acid molecule that encodes a capsid polypeptide described herein, and AAV vectors that have a capsid comprising a capsid polypeptide described herein.

### Nucleic Acid Vectors

Vectors of the present disclosure include nucleic acid vectors that comprise a polynucleotide that encodes all or a portion of a capsid polypeptide described herein. The vectors can be episomal vectors (i.e., that do not integrate into the genome of a host cell) or can be vectors that integrate into the host cell genome. Exemplary vectors that comprise a nucleic acid molecule encoding a capsid polypeptide include, but are not limited to, plasmids, cosmids, transposons and artificial chromosomes. In particular examples, the vectors are plasmids.

Vectors, such as plasmids, suitable for use in bacterial, insect and mammalian cells are widely described and well-known in the art. Those skilled in the art would appreciate that vectors of the present disclosure may also contain additional sequences and elements useful for the replication of the vector in prokaryotic and/or eukaryotic cells, selection of the vector and the expression of a heterologous sequence in a variety of host cells. For example, the vectors of the present disclosure can include a prokaryotic replicon (that is, a sequence having the ability to direct autonomous replication and maintenance of the vector extra-chromosomally in a prokaryotic host cell, such as a bacterial host cell. Such replicons are well known in the art. In some embodiments, the vectors can include a shuttle element that makes the vectors suitable for replication and integration in both prokaryotes and eukaryotes. In addition, vectors may also include a gene whose expression confers a detectable marker such as a drug resistance gene, which allows for selection and maintenance of the host cells. Vectors may also have a reportable marker, such as gene encoding a fluorescent or other detectable protein. The nucleic acid vectors will likely also comprise other elements, including any one or more of those described below. Most typically, the vectors will comprise a promoter operably linked to the nucleic acid encoding the capsid protein.

The nucleic acid vectors of the present disclosure can be constructed using known techniques, including, without limitation, the standard techniques of restriction endonuclease digestion, ligation, transformation, plasmid purification, in vitro or chemical synthesis of DNA, and DNA sequencing. The vectors of the present disclosure may be introduced into a host cell using any method known in the art. Accordingly, the present disclosure is also directed to host cells comprising a vector or nucleic acid described herein.

### AAV Vectors

Provided herein are AAV vectors comprising a capsid polypeptide described herein. Methods for vectorizing a capsid protein are well known in the art and any suitable method can be employed for the purposes of the present disclosure. For example, the cap gene can be recovered (e.g. by PCR or digest with enzymes that cut upstream and downstream of cap) and cloned into a packaging construct containing rep. Any AAV rep gene may be used, including, for example, a rep gene is from AAV1, AAV2, AAV3, AAV4, AAV5, AAV6, AAV7, AAV8, AAV9, AAV10, AAV11, AAV12 or AAV13 and any variants thereof. Typically, the cap gene is cloned downstream of rep so the rep p40 promoter can drive cap expression. This construct does not contain ITRs. This construct is then introduced into a packaging cell line with a second construct containing ITRs, typically flanking a heterologous coding sequence. Helper function or a helper virus are also introduced, and recombinant AAV comprising a capsid generated from capsid proteins expressed from the cap gene, and encapsidating a genome comprising the transgene flanked by the ITRs, is recovered from the supernatant of the packaging cell line. Various types of cells can be used as the packaging cell line. For example, packaging cell lines that can be used include, but are not limited to, HEK293 cells, HeLa cells, and Vero cells, for example as disclosed in US20110201088. The helper functions may be provided by one or more helper plasmids or helper viruses comprising adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV.

In still further instances, the AAV vectors are produced synthetically, by synthesising AAV capsid proteins and assembling and packaging the capsids in vitro.

Typically, the AAV vectors of the present disclosure also comprise a heterologous coding sequence. The heterologous coding sequence may be operably linked to a promoter to facilitate expression of the sequence. The heterologous coding sequence can encode a peptide or polypeptide, such as a therapeutic peptide or polypeptide, or can encode a polynucleotide or transcript that itself has a function or activity, such as an antisense or inhibitory oligonucleotide, including antisense DNA and RNA (e.g. miRNA, siRNA, and shRNA). In some examples, the heterologous coding sequence is a stretch of nucleic acids that is essentially homologous to a stretch of nucleic acids in the genomic DNA of an animal, such that when the heterologous coding sequence is introduced into a cell of the animal, homologous recombination between the heterologous coding sequence and the genomic DNA can occur. As would be appreciated, the nature of the heterologous coding sequence is not essential to the present disclosure. In particular embodiments, the vectors comprising the heterologous coding sequence(s) will be used in gene therapy.

In particular examples, the heterologous coding sequence encodes a peptide or polypeptide, or polynucleotide, whose expression is of therapeutic use, such as, for example, for the treatment of a disease or disorder. For example, expression of a therapeutic peptide or polypeptide may serve to restore or replace the function of the endogenous form of the peptide or polypeptide that is defective (i.e. gene replacement therapy). In other examples, expression of a therapeutic peptide or polypeptide, or polynucleotide, from the heterologous sequence serves to alter the levels and/or activity of one or more other peptides, polypeptides or polynucleotides in the host cell. Thus, according to particular embodiments, the expression of a heterologous coding sequence introduced by a vector described herein into a host cell can be used to provide a therapeutic amount of a peptide, polypeptide or polynucleotide to ameliorate the symptoms of a disease or disorder. In other instance, the heterologous coding sequence is a stretch of nucleic acids that is essentially homologous to a stretch of nucleic acids in the genomic DNA of an animal, such that when the heterologous sequence is introduced into a cell of the animal, homologous recombination between the heterologous coding sequence and the genomic DNA can occur. Accordingly, the introduction of a heterologous sequence by an AAV vector described herein into a host cell can be used to correct mutations in genomic DNA, which in turn can ameliorate the symptoms of a disease or disorder.

In non-limiting examples, the heterologous coding sequence encodes an expression product that, when delivered to a subject using an AAV vector of the present disclosure, treats a CNS-associated disease or condition (i.e. a disease or condition with a pathology that manifests at least in part in the CNS, and/or is caused at least in part by expression of one or more genes in the CNS). In one example, the heterologous coding sequence encodes an expression product that, when delivered to the retina of a subject, treats a retinal disease or condition (i.e. a disease or condition with a pathology that manifests at least in part in the retina, and/or is caused at least in part by expression of one or more genes in the retina). In illustrative embodiments, the disease or condition is selected from among Leber's congenital amaurosis, age-related macular degeneration (AMD; including wet AMD and dry AMD), retinitis pigmentosa, choroideremia, Stargardt macular degeneration, achromatopsia, X-linked retinitis pigmentosa and X-linked juvenile retinoschisis. In other examples, the heterologous coding sequence encodes an expression product that, when delivered to the brain of a subject, treats a disease or condition of the brain (i.e. a disease or condition with a pathology that manifests at least in part in the brain, and/or is caused at least in part by expression of one or more genes in the brain), or a neurodegenerative disease or condition. In illustrative embodiments, the disease or condition is selected from among X-linked adrenoleukodystrophy (ALD), spinal muscular atrophy (SMA), amyotrophic lateral sclerosis, mucopolysaccharidosis type III A and Batten disease. Those skilled in the art would readily be able to select an appropriate heterologous coding sequence useful for treating such CNS-associated diseases. In some examples, the heterologous coding sequence comprises all or a part of a gene that is associated with the disease, such as all or a part of a gene set forth in Table 2. In such instances, the AAV vector is typically used for gene replacement therapy in which a functional copy of the gene is introduced into the retina, or for genome editing (e.g. CRISPR-Cas9 based genome editing) in which one or more defective copies of a gene is corrected or ablated. In other instances, the heterologous coding sequence encodes a therapeutic protein that is not associated with the disease (i.e. is not causative of the disease in the subject), such as an anti-inflammatory agent (e.g. an anti-VEGF agent such as an anti-VEGF antibody or a VEGF trap), an anti-apoptotic agents or neurotrophic agent (e.g. EPO, CNTF, GDNF, NGF), or an anti-complement agent (e.g. CD59). These agents can be delivered to the retina for the treatment of various retinal diseases, including AMD.

TABLE 2

| Exemplary CNS-associated | Exemplary associated |
|---|---|
| disease | gene(s) |
| Leber congenital amaurosis | RPE65, GUCY2D, CEP290, |
| | CRB1 |
| Choroideremia | CHM |
| Stargardt macular degeneration | ABCA4, ELOVL4 |
| Achromatopsia | CNGB3, CNGA, GNAT2, |
| | PDE6C, PDE6H |
| X-linked retinitis pigmentosa | RPGR, RP2 |
| X-linked juvenile retinoschisis | RS1 |
| Retinitis pigmentosa | RLBP1, PDE6B, MERTK |
| X-linked adrenoleukodystrophy | ABCD1 |
| (ALD) | |
| Spinal muscular atrophy (SMA) | SMN1, SMN2 |
| Amyotrophic lateral sclerosis | SOD1 |
| Mucopolysaccharidosis type III A | SGSH |
| Batten | CLN2, CLN6, TPP1 |

The heterologous coding sequence in the AAV vector is flanked by 3′ and 5′ AAV ITRs. AAV ITRs used in the vectors of the disclosure need not have a wild-type nucleotide sequence, and may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

As will be appreciated by a skilled artisan, any method suitable for purifying AAV can be used in the embodiments described herein to purify the AAV vectors, and such methods are well known in the art. For example, the AAV vectors can be isolated and purified from packaging cells and/or the supernatant of the packaging cells. In some embodiments, the AAV is purified by separation method using a CsCl or iodixanol gradient centrifugation. In other embodiments, AAV is purified as described in US20020136710 using a solid support that includes a matrix to which an artificial receptor or receptor-like molecule that mediates AAV attachment is immobilized.

### Additional Elements in the Vectors

The vectors of the present disclosure can comprise promoters. In instances where the vector is a nucleic acid vector comprising nucleic acid encoding the capsid polypeptide, the promoter may facilitate expression of the nucleic acid encoding the capsid polypeptide. In instances where the vector is an AAV vector, the promoter may facilitate expression of a heterologous coding sequence, as described above.

In some examples, the promoters are AAV promoters, such as the p5, p19 or p40 promoter. In other examples, the promoters are derived from other sources. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Non-limiting examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system; the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In some embodiments, tissue specific promoters are used. Non-limiting examples of such promoters include retina-specific promoters such as human cone L/M-opsin (PR2.1) promoter and variants thereof (see e.g. Ye et al. 2016, Hum Gene Ther. 27(1): 72-82), the chimeric IRBPe/GNAT2 promoter, the synthetic synGNAT2/GNAT2 promoter (Dyka et al. 2014, Adv Exp Med Biol. 801: 695-701) and other synthetic retina-specific promoters such as those described by Juttner et al. 2019, Nature Neuroscience. 22:1345-1356). In other embodiments, the promoters selectively function in the brain, and may include, for example, human synapsin 1 (Syn1) promoter, neuron-specific enolase (NSE) promoter, human myelin associated (MAG) promoter (see e.g. Ingusci et a., 2019, Front. Pharmacol. 10:724. doi: 10.3389/fphar.2019.00724). The selection of an appropriate promoter is well within the ability of one of ordinary skill in the art.

The vectors can also include transcriptional enhancers, translational signals, and transcriptional and translational termination signals. Examples of transcriptional termination signals include, but are not limited to, polyadenylation signal sequences, such as bovine growth hormone (BGH) poly(A), SV40 late poly(A), rabbit beta-globin (RBG) poly(A), thymidine kinase (TK) poly(A) sequences, and any variants thereof. In some embodiments, the transcriptional termination region is located downstream of the posttranscriptional regulatory element. In some embodiments, the transcriptional termination region is a polyadenylation signal sequence.

The vectors can include various posttranscriptional regulatory elements. In some embodiments, the posttranscriptional regulatory element can be a viral posttranscriptional regulatory element. Non-limiting examples of viral posttranscriptional regulatory element include woodchuck hepatitis virus posttranscriptional regulatory element (WPRE), hepatitis B virus posttranscriptional regulatory element (HBVPRE), RNA transport element, and any variants thereof. The RTE can be a rev response element (RRE), for example, a lentiviral RRE. A non-limiting example is bovine immunodeficiency virus rev response element (RRE). In some embodiments, the RTE is a constitutive transport element (CTE). Examples of CTE include, but are not limited to, Mason-Pfizer Monkey Virus CTE and Avian Leukemia Virus CTE.

A signal peptide sequence can also be included in the vector to provide for secretion of a polypeptide from a mammalian cell. Examples of signal peptides include, but are not limited to, the endogenous signal peptide for HGH and variants thereof; the endogenous signal peptide for interferons and variants thereof, including the signal peptide of type I, II and III interferons and variants thereof; and the endogenous signal peptides for known cytokines and variants thereof, such as the signal peptide of erythropoietin (EPO), insulin, TGF-β1, TNF, IL1-α, and IL1-β, and variants thereof. Typically, the nucleotide sequence of the signal peptide is located immediately upstream of the heterologous sequence (e.g., fused at the 5′ of the coding region of the protein of interest) in the vector.

In further examples, the vectors can contain a regulatory sequence that allows, for example, the translation of multiple proteins from a single mRNA. Non-limiting examples of such regulatory sequences include internal ribosome entry site (IRES) and 2A self-processing sequence, such as a 2A peptide site from foot-and-mouth disease virus (F2A sequence).

### Host Cells

Also provided herein are host cells comprising a nucleic acid molecule or vector (including an AAV vector) or of the present disclosure. In some instances, the host cells are used to select, amplify, replicate, package and/or purify a polynucleotide or vector. In other examples, the host cells are used to express a heterologous sequence, such as one packaged within an AAV vector. Exemplary host cells include prokaryotic and eukaryotic cells. In some instances, the host cell is a mammalian host cell, such as a human host cell. It is well within the skill of a skilled artisan to select an appropriate host cell for the expression, amplification, replication, packaging and/or purification of a polynucleotide, vector or rAAV virion of the present disclosure. Exemplary mammalian host cells include cells of the CNS, including retinal cells, or cells derived from retinal cells (such as cells lines derived from retinal cells) and cells in the brain. In particular examples, the host cell is a Müller glia cell, retinal pigment epithelium cell, photoreceptor cell (rods and cones), astrocytes, horizontal cell bipolar cell, amacrine cell and/or retinal ganglion cell. Exemplary mammalian host cells also include cells, or cells derived from retinal cells (such as cells lines derived from retinal cells). In particular examples, the host cell is a Müller glia cell, retinal pigment epithelium cell, photoreceptor cell (rods and cones), astrocyte, horizontal cell bipolar cell, amacrine cell, retinal ganglion cell, neuron, oligodendrocyte, and microglia.

### Compositions and Methods

Also provided are compositions comprising the nucleic acid molecules, polypeptides and/or vectors of the present disclosure. In particular examples, provided are pharmaceutical compositions comprising the AAV vectors disclosed herein and a pharmaceutically acceptable carrier. The compositions can also comprise additional ingredients such as diluents, stabilizers, excipients, and adjuvants.

The carriers, diluents and adjuvants can include buffers such as phosphate, citrate, or other organic acids; antioxidants such as ascorbic acid; low molecular weight polypeptides (e.g., less than about 10 residues); proteins such as serum aAAVC.umin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

The AAV vectors of the present disclosure, and compositions containing the AAV vectors, may be used in methods for the introduction of a heterologous coding sequence into a host cell. Such methods involve contacting the host cell with the AAV vector. This may be performed in vitro, ex vivo or in vivo. In particular embodiments, the host cell is a retinal cell.

When the methods are performed ex vivo or in vivo, typically the introduction of the heterologous sequence into the host cell is for therapeutic purposes, whereby expression of the heterologous sequence results in the treatment of a disease or condition. Thus, the AAV vectors disclosed herein can be administered to a subject (e.g., a human) in need thereof, such as subject with a disease or condition amendable to treatment with a protein, peptide or polynucleotide encoded by a heterologous sequence described herein.

When used in vivo, titres of AAV vectors to be administered to a subject will vary depending on, for example, the particular recombinant virus, the disease or disorder to be treated, the mode of administration, the treatment goal, the individual to be treated, and the cell type(s) being targeted, and can be determined by methods well known to those skilled in the art. Although the exact dosage will be determined on an individual basis, in most cases, typically, recombinant viruses of the present disclosure can be administered to a subject at a dose of between 1×10¹⁰ genome copies of the recombinant virus per kg of the subject and 1×10¹⁴ genome copies per kg. In other examples, less than 1×10¹⁰ genome copies may be sufficient for a therapeutic effect. In other examples, more than 1×10¹⁴ genome copies may be required for a therapeutic effect.

The route of the administration is not particularly limited. For example, a therapeutically effective amount of the AAV vector can be administered to the subject via, for example, intravitreal, subretinal, intrathecal, intravenous, intraperitoneal, subcutaneous, epicutaneous, intradermal, intramuscular, pulmonary, intracranial, intraosseous, oral, buccal, or nasal routes. In particular embodiments, the AAV vector is administered to the subject via intravitreal injection. The AAV vector can be administrated as a single dose or multiple doses, and at varying intervals.

Also provided are methods for producing an AAV vector described above and herein, i.e. one comprising a capsid polypeptide of the present disclosure. Such methods comprise culturing a host cell comprising a nucleic acid molecule encoding a capsid polypeptide the present disclosure, an AAV rep gene, a heterologous coding sequence flanked by AAV inverted terminal repeats, and helper functions for generating a productive AAV infection, under conditions suitable to facilitate assembly of an AAV vector comprising a capsid comprising a capsid polypeptide of the present disclosure, wherein the capsid encapsidates the heterologous coding sequence.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1. Materials and Methods

### Retinal Explant Collection and Preparation

Following SCHN HREC and governance approvals, NSW Eye Bank ethical approval and CMRI governance approval and MTA agreement, individual donor eye globes were obtained from the NSW Eye Bank (Sydney Eye Hospital, NSW, Australia). The retina was dissected from the eye and placed in sterile Ames medium before further culture/use (described below).

Human Retina Explant Culture

Ames' medium was prepared according to manufacturers' protocol, then bubbled with carbogen (95% O₂/5% CO₂). To 1 L medium, the following was added: 10 mL penicillin/streptomycin solution (100 U/ml pen, 100 μg/ml strep mixed solution); 10 mL L-glutamine (0.292 mg/ml); and 5 mL Horse Serum (0.5%). Medium was bubbled with carbogen until use.

The carbon dioxide-independent medium was replaced with −30 mL Ames' medium in sterile specimen jars containing donor eyes. The whole retina was transferred into carboxygenated medium at room temperature. The retina was separated into quadrants and each quadrant separated into a central (A) and peripheral (B) portion and cultured on separate membranes. The location of retinal pieces was tracked. Each piece of retina was approximately 0.5×2 cm, rectangular in shape, to ensure optimal cell numbers during cell sorting process. Alternatively, 5 to 8 mm trephines (using a biopsy punch) were taken from areas of interest (macula, periphery, mid-periphery).

Free-Floating Retinal Explant Immersion Method

During Round 1 selection (described below) the “Free-floating Retinal Explant Immersion method” of culture was used. Human retinal explants were obtained as described above, with 5 and 8 mm trephines taken from areas of interest (macular, mid-periphery, periphery). 5 mm pieces were taken for use as controls (negative and positive). 8 mm pieces were used for carrying out first round AAV library selection. The retinal explants (5 or 8 mm) were incubated as free-floating tissue pieces in 1 mL of Ames media in 24-well plate for 16 hr, 37° C., 5% CO₂.

Interphase Retinal Explant Culture System

Donor eyes were obtained and prepared as retinal explant cultures as described above, with trephines taken from areas of interest (macular, mid-periphery, periphery). During Round 2 selection (described below) the “Interphase Retinal Explant Culture System” was used (Retinal ganglion cell side up, photoreceptors down), to simulate intravitreal injection and with the aim to select for photoreceptors.

Four retinal tissue pieces, each of approximately 1×2 cm² in size, were dissected out and placed onto the prepared tissue culture insert for second round AAV library selection. In addition, 8 mm trephine pieces were taken for use as control stains (negative and positive). A tissue culture insert (0.4 μm pore size, 30 mm diameter; Merck-Millipore) was prepared for each retinal piece. The insert was filled with media then the retinal piece was transferred onto the insert, ganglion cell side up. A pipette was used to remove the media from inside the membrane.

A gentle suction was applied (for ˜30 sec) to the underside of the membrane to allow attachment of the tissue. This was done by placing a piece of filter paper underneath the membrane, and the retina was kept on membrane. Cell culture dishes (100 mm diameter, 20 mm depth) were filled with 65 ml of Ames' medium (containing 0.192% sodium bicarbonate, 100 U/mL penicillin, 100 mg/mL streptomycin, 0.5% Horse Serum, and 0.292 mg/mL L-glutamine) and the tissue culture insert rested on the custom printed filter stands (polylactic acid (PLA) polymer using a 3D-printer at WIMR, Dr Suat Dervish) and then placed into the cell culture dish, ensuring the retina was contacted with the medium via the filter on the photoreceptor side and with the incubator atmosphere (5% CO₂, 95% air, 37° C., humidified) on the ganglion cell side. The photoreceptor side of the retinal explant faced down towards the media, while the ganglion cell layer side faced the top and was exposed to air. These were used for carrying out second round AAV library selection on. Retinal explants were incubated as larger tissue pieces (1×2 cm²) on the well insert and stand, and the free-floating tissue pieces (8 mm) in 1 mL of Ames media in 24-well plate, for 16 hr, 37° C., 5% CO₂.

### AAV2 Library

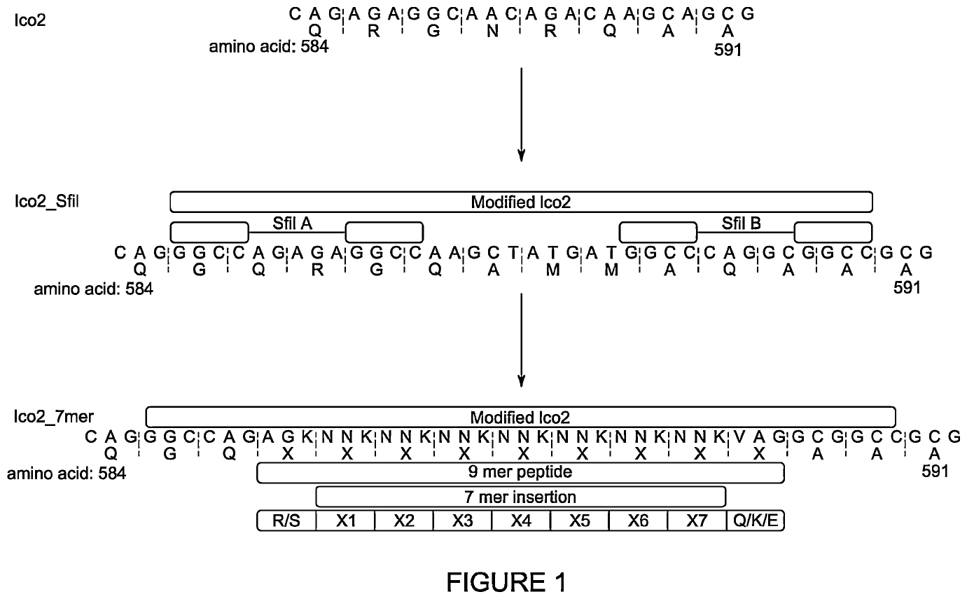

An AAV2 peptide library (FT-SFFV-GFP.AAVLib^{2-PEP}) with a titre of 1.3×10¹³ vg/mL a complexity of 4.2×10⁶ unique variants was utilised. This library contained capsid variants that were based on AAV2 (SEQ ID NO:1), modified to incorporate restriction sites allowing insertion of 7 randomised amino acids forming the core of the polypeptide inserted after VP1-N587 into variable region VIII. The two flanking amino acids of the 9 amino acid peptides were only semi-variable as a result of the cloning strategy and contained either an arginine or serine as the start of the peptide and were terminated by either a glutamine, lysine or glutamate (see FIG. 1). The constructs contained eGFP for sorting and selection of highly transduced cells.

For the preparation of the AAV2 peptide display library, double SfiI restriction sites were inserted into the local codon-optimized version of the AAV2 cap gene (CapIco2) at the N587 insertion site, as described previously (Logan et al., 2017, Nat Genet 49: 1267). In brief, using primers with very long overhangs and 18 nt homology arms at the 3′end between each other, the pRep2CapIco2 plasmid was amplified and the added regions (primer overhangs bearing SfiI sites) were incorporated using NEBuilder (NEB, Cat #E2621). The resulting plasmid pRep2CapIco2_SfiI was then digested using SwaI and NsiI, and the capsid-containing fragment was ligated into the equally SwaI/NsiI digested FT-SFFV selection platform. This FT-SFFV-Ico2_SfiI construct was now digested with SfiI overnight, purified using the QIAquick PCR Purification Kit (QIAGEN, Cat #28104), and re-digested with SfiI overnight. Lastly, the construct was dephosphorylated using calf intestinal alkaline phosphatase (NEB, Cat #M0290). Following electrophoretic separation and gel extraction (Zymogen, Cat #D4001), the backbone was ready for accepting the peptide library-coding fragment.

The peptide library itself was ordered as an oligo nucleotide with 20 nt homologies to each end of the SfiI-digested FT-SFFV-Ico2_SfiI backbone flanking a NNK7 motif coding for randomized amino acids with lower redundancy (Müller et al. 2003, Nat Biotechnol 21(9): 1040-1046). In addition to the seven random amino acids, the ‘SfiI-clipped’ codons upstream (arginine or serine) and downstream (glutamine, lysine or glutamate) of the random insertion were coded to be semi-variable (full oligo as ordered in reverse complement, Ico2_NNK7). Therefore, the library contained a 7-mer random insert flanked by two variable amino acids coding for 9-mer novel peptides. Before insertion into the backbone, the oligonucleotide was made double stranded using a short primer binding on the homology arm upstream of the peptide (Ico2-dsSyn), Klenow (exo-) (NEB, Cat #M0212), and dNTPs (NEB, Cat #N0447). The fragment (dsIco2-library) was gel purified and ready for insertion.

The final library was generated by mixing 225 fmol of the digested FT-SFFV-Ico2_SfiI backbone with 2250 fmol of the dsIco2-library insert into 13 individual NEBuilder (NEB, Cat #E2621) reactions. The reactions were combined after assembly and purified using ethanol precipitation. The resulting pellet (1 μg of DNA) was used for electroporation into competent cells (Lucigen, Cat #60512). The recovered transformants were used to inoculate 250 mL lysogeny broth (LB) containing 10 μg/mL trimethoprim (TMP). Only 10 μL of recovered transformants were used to plate a 10⁻¹-10⁻⁵ dilution series on TMP-LB-agar plates to determine transformation efficiency (2.3×10⁷ colonies per μg of DNA). The 250 mL of inculcated LB were maxi prepped (Invitrogen, Cat #A31217).

### Library Selection—Round 1

Round 1 selection of the FT-SFFV-GFP.AAVLib^{2-PEP} library was performed on primary human retina explants (Free-floating Retinal Explants). The CsCl purified library was added to retina explants floating in media (Free-floating Retinal Explant Immersion method).

The library was added at varying volumes (5, 10, 20 and 25 μL [corresponding to doses: 6.5×10¹⁰, 1.3×10¹¹, 2.6×10¹¹ and 3.3×10¹¹ vector genome copies per well, respectively]) to each of the 8 mm retinal explants (×4) and incubated for further 5 days, at 37 PC, 5% CO₂. Media change was performed every 24 hr (Selection Round 1). For one of the 8 mm retinal explants, AAV5-CMV-GFP (5.10×10¹⁰ GC per well) was added for single stain GFP control. Two other 5 mm retinal explants were used for no stain and CD73-APC single stain control. Cells were incubated for 7 days, 37° C., 5% CO₂, with media changed every 24 hr.

Media was removed from each well and the retinal explants transferred to a petri dish with minimal Ames/DMEM medium. Retinal explants were chopped into fine pieces with a scalpel before being transferred to a 1.5 mL Eppendorf tube with 500 uL digestion buffer (2.5 mg/mL papain, 10 mg/mL DNase, 1.5 μg/mL superoxide dismutase in HBSS with 5 mM MgCl₂) and digested for 5 mins at 37° C. The temperature was lowered to 8° C. and maintained for 20 mins before the tube was centrifuged 300×g for 5 min at 4° C. and supernatant removed. The pellet was resuspended in 500 uL neutralisation buffer (50 μg/mL antipain, 10 mg/mL DNase, 1.5 μg/mL superoxide dismutase in HBSS with 5 mM MgCl₂) and incubated for 10 min on ice. Aliquots of 50-100 uL were taken for negative and single stain controls at this time. Samples were centrifuged at 300×g for 5 min at 4° C. and the supernatant removed and discarded.

To stain cells with CD73-APC conjugate antibody (1:10), the pellet was resuspended in staining buffer (10 mg/mL BSA, 10 mg/mL DNase, in PBS with 5 mM MgCl₂) containing the antibody and incubated at 4° C. for 40 min. For unstained controls, resuspend pellet in PBS and incubate at 4° C. for 40 min. For FACS analysis, cells were again centrifuged, washed in PBS and then resuspended in FACS buffer (2% FCS, 10 mg/mL DNase, in PBS with 5 mM MgCl₂, 0.22 μm filtered).

A GFP⁺/CD73⁺ population from each of the four conditions (i.e. each of the different amounts of library added to the explants) was identified and pooled with an unsorted population. RNA and DNA was extracted, cDNA produced and then cDNA and DNA sequenced using Next Generation Sequencing (NGS).

### Library Selection—Round 2

The secondary library (enriched FT-SFFV-GFP.AAVLib^{2-PEP} library from first round of selection) was produced and packaged as described above but purified using iodixanol gradient ultracentrifugation. 1×10¹¹ (L), 3×10¹¹ (M) and 6×10¹¹ (H) vg was used to transduce retinal explants cultured using the interphase culture system and transduced from the side of the ganglion cell layer.

The second FT-SFFV-GFP.AAVLib^{2-PEP} library was added at varying volumes (18.5, 37, 75, and 150 μL [corresponding to doses: 7.4×10¹⁰, 1.5×10¹¹, 3.0×10¹¹ and 6.0×10¹¹ vector genome copies per well, respectively]) to each of the larger retinal tissue pieces (1×2 cm²), on top of the ganglion cell layer side, and incubated for 6 days, 37° C., 5% CO₂, with Ames' media change every 24 hr.

For the 8 mm retinal explants, AAV7m8-CMV-GFP (1.0×10¹⁰ GC per well) was added for single stain GFP control. There were two other conditions for the 8 mm retinal explants; no stain control and CD73-APC single stain control. The tissue was incubated for further 4 days, 37° C., 5% CO₂, with a media change every 24 hr.

Four days post transduction tissues were harvested, dissociated, labelled for CD73 surface receptor (photoreceptor marker in mature photoreceptors) and analysed for GFP expression. CD73-positive cells were sorted for conditions M and H and 5% GFP positive CD73 cells were detected in those conditions. Cells transduced with the lowest dose of AAV (L) were not sorted. RNA was extracted from non-sorted (L) or CD73-positive cells (M/H) using trizol-isopropanol precipitation and processed for cDNA synthesis as described above. cDNA was amplified for NGS analysis.

Cells were prepared for FACS analysis as described above and sorted for GFP⁺/CD73-APC⁺ cells. For each of the conditions, 18.5, 37, 75, and 150 μL, the % of GFP⁺/CD73-APC⁺ cells was 0.7, 0.8, 1.8 and 1.7, respectively. CD73+ population from the 75 uL, 150 uL conditions, as well as unsorted populations, were separately sorted into 100% FCS. Subsequent DNA and RNA extraction was performed and NGS done. Candidate capsids from this Round 2 selection were then packaged and titred as a library for third round validation.

Maintenance of Human Induced Pluripotent Stem (iPS) Cells

Human induced pluripotent stem (iPS) cell lines was maintained on feeder-free conditions on Essential 8 media (E8, Life Technologies) and Geltrex (Life Technologies) coated 6 well plates. When 70% confluent, these cells were dissociated using Versene solution (Life Technologies) at 37° C. for 5 to 10 minutes. The dissociated clumps were washed once in PBS with centrifugation at 900 Revolutions per minute (RPM) for 5 minutes. The resulting cell pellet was further dissociated and resuspended in 1 ml E8 media and between 70-100 ul of cells was added to each well of the 6 well plate containing 2 ml E8 and 10 μM ROCK inhibitor (Y-27632 dihydrochloride, Tocris) for 24 hours. Daily feeding with E8 was continued for further maintenance culture.

### Retinal Organoid Differentiation Culture

To generate retinal organoids, human iPS cells were maintained as described above until 90-95% confluent. Essential 6 media (E6, Life Technologies) was added to the cultures for 2 days (replaced fresh between day 1 and 2 of differentiation). At day 3 of differentiation media was replaced with a pro-neural induction media (PIM, composed of Advanced DMEM/F12, N2 supplement, L-Glut, non-essential amino acid and Antibiotic-antimycotic). Neuroretinal vesicles started to appear between weeks 4 and 7 of culture. They are discerned by the typical transparent neural epithelium that arises from islands of pigmented retinal pigmented epithelium cells. During this period they were manually excised with 19G needles and kept individually in low binding 96 well plates (Nunclon™ 43ector, ThermoFisher) to mature into retinal organoids and maintained in retinal differentiation media (RDM composed of DMEM, F12 Nutrient mix, B27 without retinoic acid, Antibiotic-antimycotic). At 6 weeks of differentiation, media was replaced with RDMF (RDM supplemented with 10% FBS, 100 μM taurine (Sigma, T4871) and 2 mM glutamax). At 10 weeks, RDMF was replaced by ALT70 media (composed of Advanced DMEM/F12 supplemented with all the components of RDMF and 1 μM retinoic acid (RA)) and retinal organoids were transferred to low binding 24 well plates. At 12 weeks of differentiation, ALT70 media was replaced with ALT90 media (Alt70 media supplemented with 1% N2 supplement and reduced RA concentration of 0.5 μM). All media described here were replaced every Monday, Wednesday and Friday.

### Brain Cortical Organoid Differentiation Culture

The same protocol that gives rise to Neuroretinal vesicles also generates brain cortical organoids. As described above, human iPSCs were maintained as described above until 90-95% confluent. Essential 6 media (E6, Life Technologies) was added to the cultures for 2 days (replaced fresh between day 1 and 2 of differentiation). At day 3 of differentiation media was replaced with a pro-neural induction media (PIM, composed of Advanced DMEM/F12, N2 supplement, L-Glut, non-essential amino acid and Antibiotic-antimycotic). At around week 3-4 three-dimensional (3D) rosettes containing organoids were observed throughout the plate and in close proximity to neuroretinal vesicles. The 3D cortical organoids, were manually excised with 19G needles and kept together in 60 mm well plates in retinal differentiation media (RDM, composed of DMEM, F12 Nutrient mix, B27-vitamin A and antibiotic-antimycotic) and put on an orbital shaker at 85 RPM. At 6 weeks of differentiation, retinal differentiation medium was supplemented with 10% FBS, 100 μM Taurine (Sigma, T4871) and 2 mM Glutamax. At 10 weeks, the cortical organoids were cultured in a cerebral differentiation medium as described in Lancaster & Knoblich. Nature Protocols, 2015 (Composed of Neurobasal medium, DMEM/F12, N2 supplement, insulin, Glutamax, MEM-NEAA, B-mercaptoethanol, B27 supplement) or BrainPhys™ hPSC Neuron Kit (Stemcell Technologies, composed of BrainPhys Neuronal medium, NeuroCult SM1 neuronal supplement, N2 supplement, human recombinant brain-derived neurotrophic factor, human recombinant glial cell line-derived neurotrophic factor, ascorbic acid, dibutyryl-cAMP)

### Whole Brain Differentiation Culture

Whole-brain organoids were generated using a protocol adapted from Lancaster & Knoblich Nat. Prot. 2014. Human iPSCs line (HPSI0214i-kucg_2) was dissociated using Accutase (Life Technologies, 00-4555-56) and resuspended at 9000 cell/150 μl in Essential 6 media (E6, Life Technologies) with 4 ng/ml bFGF and 50 μM Rock inhibitor (RI; Y-27632 dihydrochloride, Tocris). On day 0, 9000 cells per well were plated in low binding 96 well U-bottom plates (Nunclon™ 44ector, ThermoFisher). On Day 2, half of the old media was replaced with 150 μl of E6 supplemented with 4 ng/ml bFGF and 50 μM RI. On Day 4, old media was replaced with E6 without bFGF and RI. On Day 6, healthy Ebs with a diameter of more than 500 μM and translucent edges were transferred to low binding 24-well ultralow attachment plates (Costar, Corning) in 500 μl of proneural induction medium (PIM composed of Advanced DMEM/F12, N2 supplement, L-Glutamine, non-essential amino acid and Antibiotic-antimycotic). Two Ebs were transferred per well of low binding 24 well plates. On Day 8, an additional 500 μl of PIM was added. On Day 10, Ebs with neuroepithelia were selected and a maximum of 16 EBs were transferred per 60 mm dish. Five ml of Cerebral differentiation medium (IDM-A; Composed of Neurobasal medium, DMEM/F12, N2 supplement, insulin, Glutamax, MEM-NEAA, B-mercaptoethanol, B27 supplement without retinoic acid) as described in Lancaster & Knoblich. Nat. Prot. 2015 was added and incubated at 37° C. On Day 12, old media was replaced with fresh IDM-A. On Day 14, media was replaced with IDM+A media (Composed of Neurobasal medium, DMEM/F12, N2 supplement, insulin, Glutamax, MEM-NEAA, B-mercaptoethanol, B27 supplement) and the dish was put on an orbital shaker with speed set at 85 RPM. Whole-brain organoids were fed every 3-4 days.

### AAV Transduction of Brain Organoids In Vitro

AAV viral vectors (1-3.6×10¹¹ vg/organoid) were added to a total volume of 375 μL using fresh IDM+A media used to culture the cortical and whole brain organoids. The organoids (2-4) were then transferred to low binding 24 well plates (Costar, Corning) and media was completely replaced with IDMA containing the AAV vectors. Cortical and whole brain organoids were incubated at 37° C. for half a day before adding another 625 μl of fresh media. After overnight culture at 37° C., the organoids and IDM+A/vector mixture were transferred to a 60 mm dish. The dish was topped up to 3 mls with fresh IDM+A media and put on an orbital shaker at 85 RPM at 37° C. After 48 hours, organoids were fed every Monday, Wednesday and Friday.

### AAV Transduction of Retinal Organoids In Vitro

AAV viral vectors (2×10¹⁰ vg/organoid) were added to a volume of 300 μl using fresh ALT90 media used to culture the retinal organoids. The organoids (2-4) were then transferred to low binding 24 well plates (Costar, Corning) and media was completely replaced with ALT90 containing the AAV vectors. Organoids were incubated at 37° C. for half a day before adding another 700 μl of fresh media. After overnight incubation at 37° C., an additional 1 ml of ALT90 media was added to retinal organoids. After 48 hours at 37° C., organoids were fed every Monday, Wednesday and Friday.

### Example 2. Generation and Assessment of Novel Capsids

Previously-undescribed AAV capsids that, when vectorised, have the ability to transduce cells of the CNS, including cells in the retina and/or brain, were identified in the studies described herein. An AAV2-based peptide library was initially subject to selection using a retinal explant culture system that was, in some aspects, designed to simulate intravitreal delivery. The properties of the identified capsids were then further validated in retinal organoids, an alternative and well-accepted system for assessing transduction of the human retina, as well as retinal explants. To further characterise the properties of the capsids, their ability to transduce other cells of the CNS, and in particular human brain cells, was assessed in brain organoids.

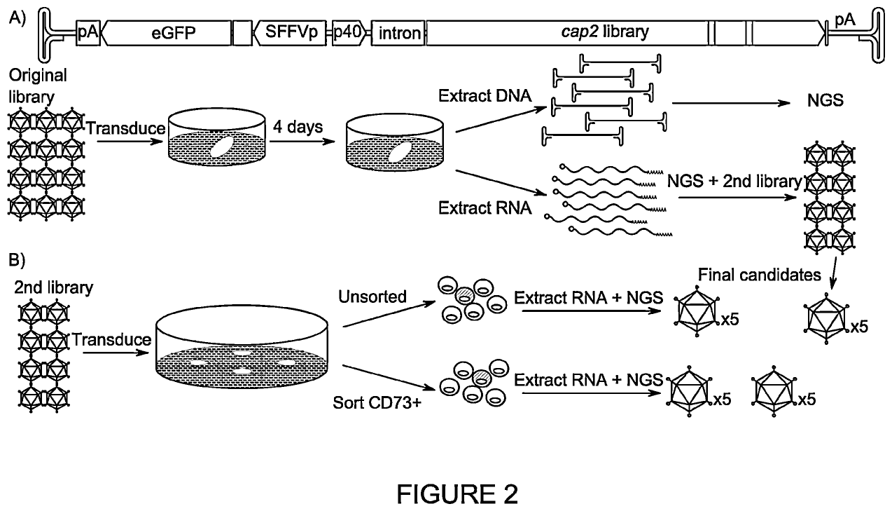

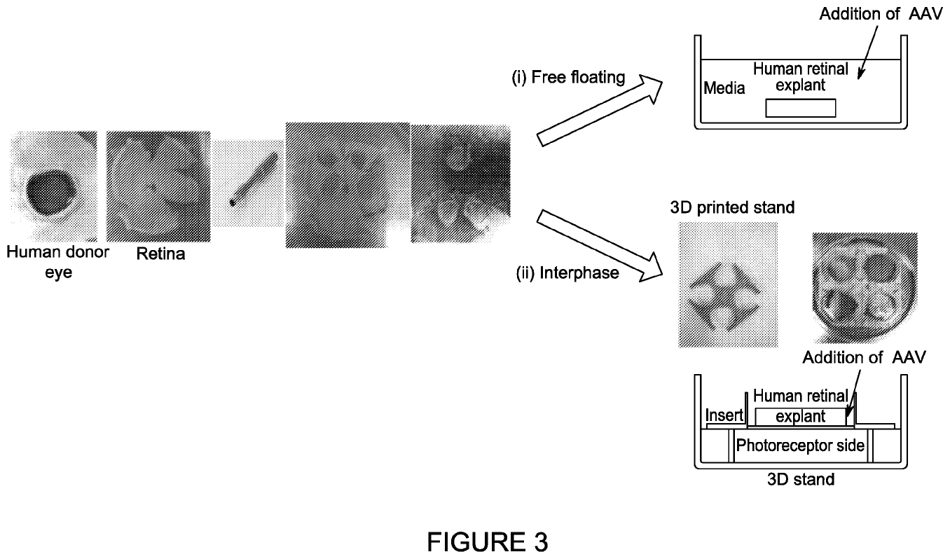

The AAV2-based peptide library was produced using an array of randomised 21 nt oligonucleotides (7×NNK-codon) inserted into variable region 8 (VRVIII) of the capsid gene, also known as loop 4 (FIG. 1). This resulted in a packaged AAV library with a minimum of 4.2 million different peptide variants, 2.9% of which contained stops codon from a theoretical maximum of 14%. The AAV2 peptide library was subjected to functional transduction (FT) selection where recovery is based on RNA (FIG. 2). In the first round of selection (Round 1, as described above) ‘Floating’ non-polarised retina explants were transduced and harvested without sorting. This step mainly removed non-functional variants from the library. The second round of selection (Round 2, as described above) was performed with a library based on the recovered variants from Round 1 and was performed using interphase retinal explants to mimic the requirement of AAV traversing the retina before reaching the photoreceptors. Table 3 sets forth the counts and percentages of the top 5 recovered variants in Round 1, and leading variants recovered in round 2 (among the unsorted population in the low dose group, the CD73+ populations in the middle and high dose groups).

TABLE 3

| | | | |
|---|---|---|---|
| Variant | | | |
| name | 9-mer Peptide | Counts | Percentage |

Round 1

| | | | |
|---|---|---|---|
| 1.1 | SAPPRHPSE | 25658 | 1.50% |
| 1.2 | RTTQFHPPE | 19916 | 1.17% |
| 1.3 | RPKQPTQPK | 13524 | 0.79% |
| 1.4 | RPSYSPSNQ | 13341 | 0.78% |
| 1.5 | SSVVSSRCE | 13163 | 0.77% |

Round 2 - Unsorted; low dose

| | | | |
|---|---|---|---|
| L1 | RKNKDTPVK | 51022 | 2.21% |
| L2 | RNQNETKRQ | 41336 | 1.79% |
| L3 | SKQLPTNNK | 39001 | 1.69% |
| L4 | RQNPKLGSE | 33414 | 1.44% |
| L5 | SKINPNASK | 27632 | 1.19% |
| M4 | SLPRRDAPK | 27304 | 1.18% |
| L6 | STRNPARNQ | 25928 | 1.12% |
| L7 | RSMGRGLGE | 25240 | 1.09% |

Round 2 - CD73⁺; medium dose

| | | | |
|---|---|---|---|
| L1 | RKNKDTPVK | 89900 | 2.94% |
| M1 | RRQRIPGGE | 86102 | 2.81% |
| M2 | SNHHTNNPK | 78538 | 2.56% |
| M3 | RNYGRQDSQ | 71595 | 2.34% |
| M4 | SLPRRDAPK | 69491 | 2.27% |
| M5 | SLDKKNATK | 69108 | 2.26% |
| L2 | RNQNETKRQ | 60017 | 1.96% |
| L3 | SKQLPTNNK | 50421 | 1.65% |
| M6 | SSQRLPTTQ | 49495 | 1.62% |
| M7 | RPTKHLDRE | 48752 | 1.59% |
| H6 | RRKAENQMK | 45759 | 1.49% |
| L5 | SKINPNASK | 41970 | 1.37% |

Round 2 - CD73⁺; high dose

| | | | |
|---|---|---|---|
| H1 | RQQPQNTRQ | 228671 | 10.29% |
| H2 | STLQRTMAK | 128948 | 5.80% |
| H3 | RHLAVAPPQ | 127910 | 5.75% |
| H4 | RPWRESSQE | 126922 | 5.71% |
| H5 | STTTRDMPK | 126428 | 5.69% |
| H6 | RRKAENQMK | 119396 | 5.37% |
| H7 | RRGSDPVRK | 118018 | 5.31% |
| H9 | RKKNEETKK | 113619 | 5.11% |
| H11 | RRINMATGQ | 111675 | 5.02% |
| L1 | RKNKDTPVK | 107193 | 4.82% |

Top candidates from the 2 rounds of selection and from different selection conditions were then selected for further analysis. The candidates, as well as benchmarks (AAV2, AAV8, AAV13, AAV 7m8, AAV Anc80, were produced by packaging a CMV-GFP-BC-WPRE-pA construct with two unique barcodes per capsid. The candidates included the top 5 candidates from Round 1 (unsorted; AAV2 1-1, AAV2 1-2, AAV2 1-3, AAV2 1-4, and AAV2 1-5); top 5 candidates from the low dose (L) group in Round 2 (not sorted for CD73⁺; AAV2 L1, AAV2 L2, AAV2 L3, AAV2 L4 and AAV2 L5; top 5 candidates from the medium dose (M) group in Round 2 (sorted for CD73⁺; AAV2 M1, AAV2 M2, AAV2 M3, AAV2 M4 and AAV2 M5; and top 5 candidates from the high dose (H) group in Round 2 (sorted for CD73⁺; AAV2 H1, AAV2 H2, AAV2 H3, AAV2 H4 and AAV2 H5) (sequences set forth in Table 4, below).

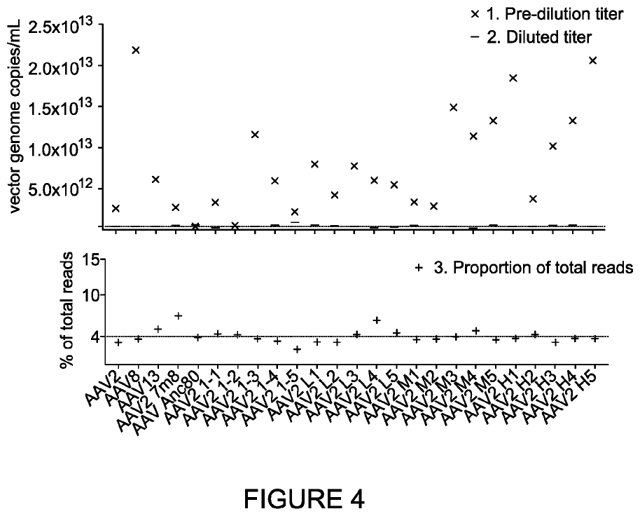

To produce the “25 Retina AAV Kit”, which contained the 20 novel variants and the 5 known AAV, the vectors were individually purified using iodixanol gradient centrifugation and titred using ddPCR (Bio-Rad, Berkeley) using QX200 ddPCR EvaGreen Supermix (Cat #1864034; Bio-Rad) with eGFP primers. The AAV were then diluted, titred with ddPCR again, mixed at equimolar ratio, concentrated using Amicon Ultra-4 Centrifuge Filter Units with Ultracef-100 kDa membrane (Cat #UFC810024; EMD Millipore) and titred one last time. As shown in FIG. 4, all novel variants apart from AAV2 1-2 and 1-5 packaged with higher efficiency than AAV2. After dilution of the AAV to 4×10¹¹ vg/mL, the AAV were mixed at equimolar ratio and the resulting mix showed a reasonable distribution. Only AAV 7m8 and AAV2-L4 were overrepresented (FIG. 4), which was taken into account in subsequent studies and ‘down’ normalised for NGS analysis.

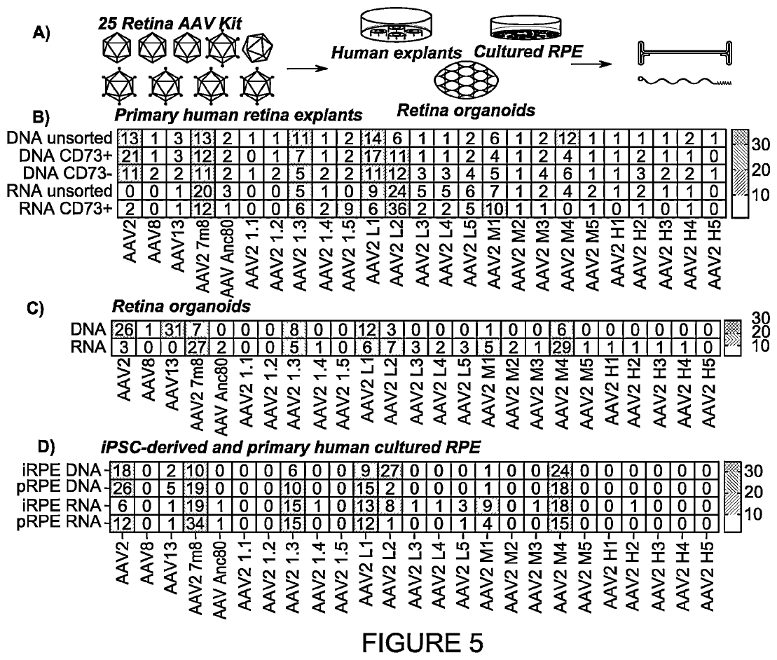

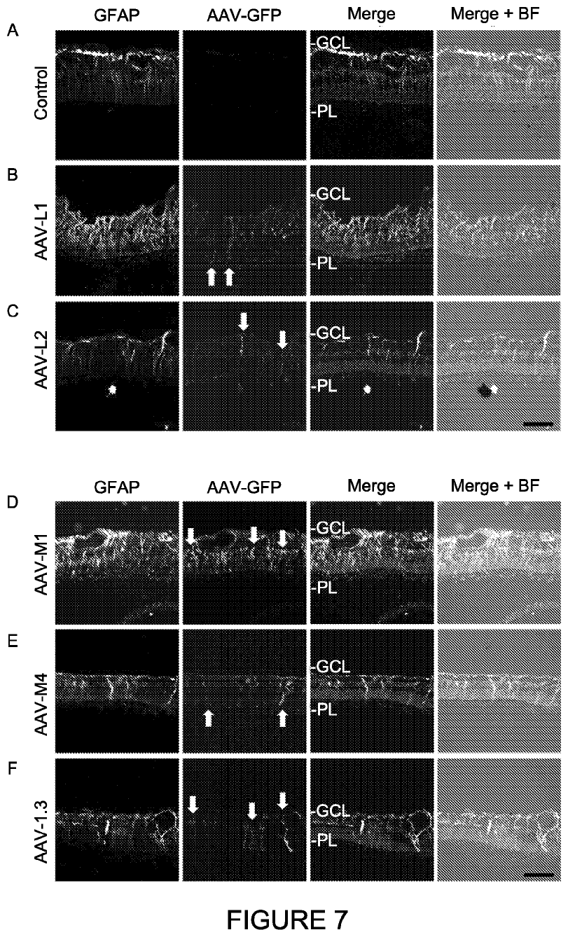

The 25 Retina AAV Kit was then used to transduce primary polarized retina explants using the “Interphase Retinal Explant Culture System”, described above, which is characterised by inner/outer retinal polarity and therefore simulates intravitreal eye injection. “25 Retina AAV kit” was added to each of the larger tissue pieces (1×2 cm²; 20 μL per piece), on top of the ganglion cell layer side, and incubated for 5 days, 37° C., 5% CO₂, with a change of media every day. Tissue was then prepared for cell sorting and the CD73⁺ population sorted into 100% FCS. Populations of unsorted cells and CD73⁻ cells were also obtained DNA and RNA was extracted and the contribution of each capsid was established by NGS. As shown in FIG. 5B, AAV2-L2 was the lead candidate in the two categories most relevant for gene therapy use: overall RNA and RNA from CD73⁺ cells, which indicate that AAV2 L2 results in the highest expression in the target cells when analysed as a whole. The RNA NGS counts of AAV2-L2 were higher than AAV 7m8, even before AAV 7m8 values were ‘down’-normalised to account for the overrepresentation in the original mix. A retinal organoid system was also used to assess transduction efficiency of the vectors, and in this system AAV2-M4 appeared to be the most effective (FIG. 5C). AAV2-M4 was also very effective at transducing iPSC-derived and primary retinal pigment epithelium (RPE) (FIG. 5D). Other AAV variants that exhibited particularly good transduction profiles in the various retinal systems included AAV2-L1, AAV2-M1 and AAV2-1.3. Immunohistochemical (IHC) analysis of retinal explants was also performed with select variants, which indicated that different vectors appear predominantly expressed in some cell types more than others (FIG. 7).

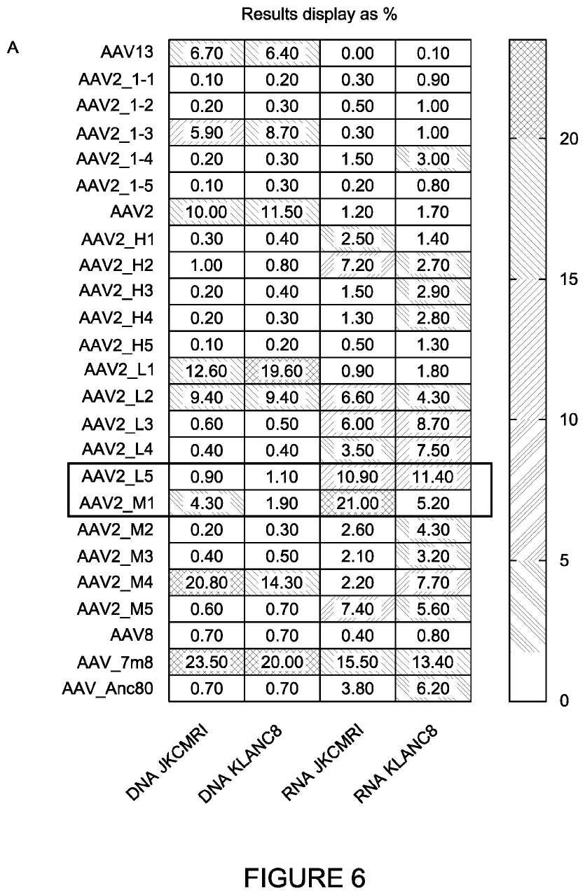

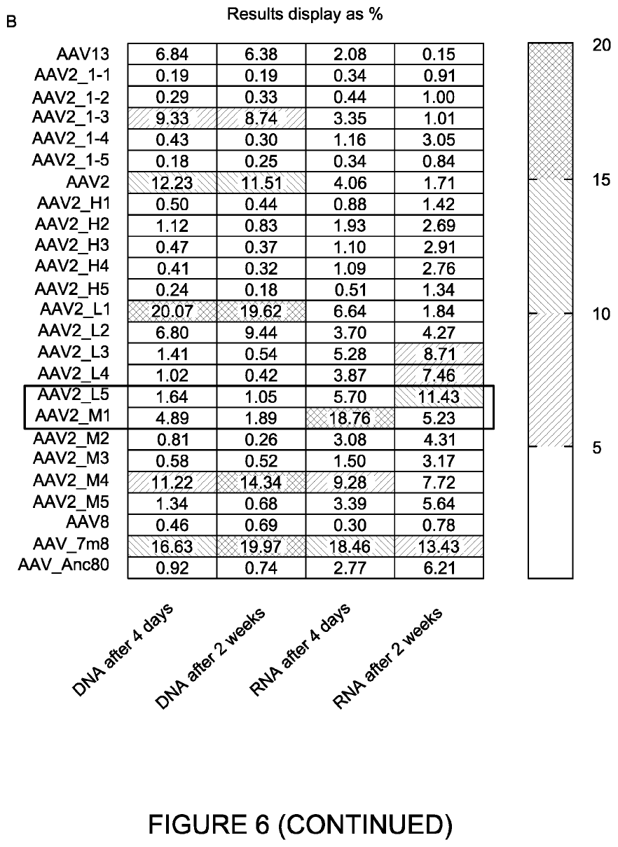

The AAV variants were also assessed for their ability to transduce brain organoids, and as shown in FIG. 6, several of the variants, and in particular, AAV2-L5 and AAV2-M1, were effective in doing so.

Further IHC analysis was conducted using retinal explants by way of validation. Human retinal explants were obtained and samples prepared using the Interphase Retinal Explant Culture System as described in Example 1. Novel AAV capsids (AAV2-L1, AAV2-L2, AAV2-M1, AAV2-M4, AAV2-1.3, AAV2-1.5) or control AAVs (AAV2, AAV8, AAV7m8) were added to each retinal tissue piece (5×10¹⁰ GC per piece). Samples were incubated for a further 3 days, 37° C., 5% COP. Media was changed every 24 hr. At the end of the incubation period, retinal explants were fixed in 4% paraformaldehyde for 1 hr, 4° C., and washed in PBS. Retinal pieces were transferred, using blunt forceps, to a 24-well plate filled with 20% sucrose, at 4° C., and incubated until the tissue sank. The solution was replaced with 30% sucrose and tissue was incubated at 4° C., until the tissue sank. The solution was changed to 2:1 30% sucrose:OCT and the tissue was incubated for 2 hr at 4° C., or until the tissue sank.

The tissue was embedded in 1:1 sucrose:OCT solution over dry ice, the solution becoming opaque as it set. Tissue was sectioned at 14 μm thickness onto adhesive slides and stored at −80° C. Sections were then permeabilised with 0.1% Triton X-100/PBS for 30 min at room temperature. Sections were blocked with 5% bovine serum albumin (BSA)/0.1% Triton X-100/PBS for 30 min at room temperature. The blocking solution was removed and primary antibody, diluted in 1% bovine serum albumin (BSA)/0.1% Triton X-100/PBS, was applied (250 μL per slide) and slides were incubated in a humidified chamber at 4° C., 16 hr. Goat anti-cone arrestin antibodies (Abcam) was used to label cone photoreceptor cells. Rabbit anti-recoverin (Merck Millipore) and anti-vimentin (ThermoFisher) antibodies were used to label rod photoreceptor and Muller glia cells, respectively. Mouse anti-PKC-α (Invitrogen) antibodies were used to label rod bipolar cells. Rabbit anti-GFAP antibodies(Sigma-Aldrich) were used to label astrocytes and retinal ganglion cells.

Slides were washed in PBS (3×5 min). Secondary antibody solution, conjugated with AlexaFluor 488, 594, or 633, was applied (1:500) and slides were incubated at room temperature for 2 hr before being washed again in PBS (3×5 min). Nuclei was counterstained with DAPI (1:10,000 in PBS) at room temperature, 20 min and then washed in PBS (3×5 min). Slides were mounted with glass coverslips with 1:1 glycerol:PBS and stored at −20° C. until ready to image using a laser confocal microscope. Results indicate that AAV2-L1, AAV2-L2, and AAV2-1.3, transduce Muller glia, rod bipolar cells and some photoreceptors. AAV2-M1 and AAV2-M4 transduce Muller glia cells very well and potentially also bipolar cells. Following anti-GFP labelling, more photoreceptors positive for GFP were weakly detected in AAV2-L1 and AAV2-L2, while AAV2-M1 showed strong GFP expression in Muller glia cells.

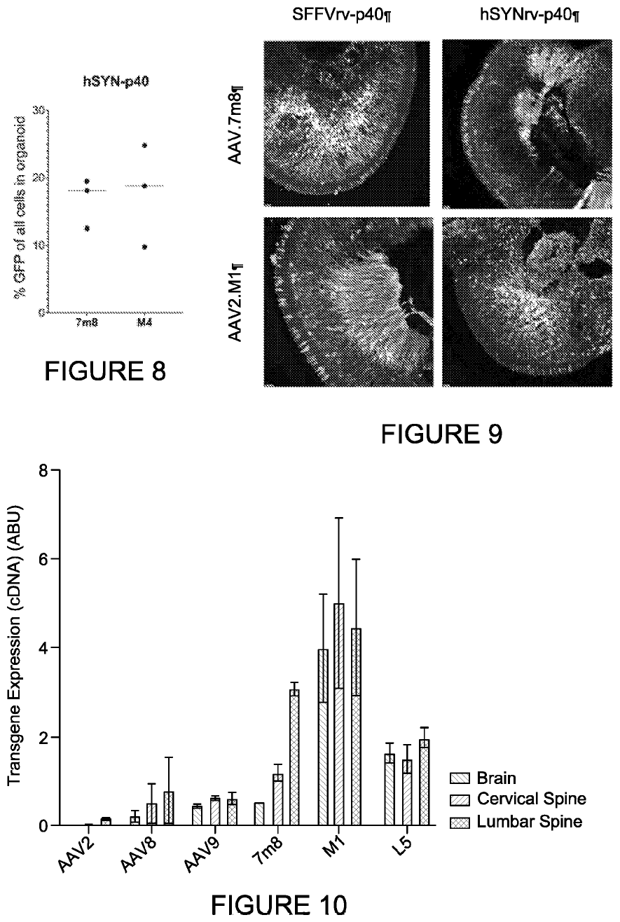

Expression of GFP was assessed in retinal organoids transduced with AAV 7m8, AAV2-M1 or AAV2-M4. In one experiment, a human synapsinl promoter was coupled, in reverse orientation, to the AAV-p40 promoter driving GFP. This configuration produces good expression in neuronal cells and reduces expression in glial cells. The experiment was performed in three independently differentiated iPSC-derived retinal organoids. Flow cytometry analysis (FIG. 8) demonstrated that expression was comparable with AAV 7m8 and AAV2-M4. Similarly, in retinal organoids transduced with AAV 7m8 or AAV2-M1 the SFFV promoter or hSYN promoter was coupled, in reverse orientation, to the AAV-p40 promoter driving GFP. IHC staining (FIG. 9) showed strong transduction of photoreceptors with both constructs, and apparently stronger expression of GFP with the AAV2-M1 capsid than with AAV 7m8 using both SFFVrv-p40 and hSYNrv-p40 promoter constructs.

A kit comprising a mixture of known natural capsids (AAV2, AAV8, AAV9), known bioengineered capsids (including AAV 7m8, LK03, N496D, N582S, R588I and R588T) and novel capsids of the present disclosure (AAV2-M1 and AAV2-L5) was prepared as described in Westhaus et al., 2019, Human Gene Therapy 31:575-589. Briefly, each AAV variant was produced with two unique identifying barcodes and purified using an Iodixanol gradient. Purified individual vectors were then titered and mixed together at an equal ratio. The final AAV mix was then analysed by NGS to confirm the ratio of the individual vectors in the AAV mix (called the ‘pre-mix’). Post-injection NGS data is normalised to the pre-mix to determine vector efficiency.

The AAV kit was injected intrathecally into an adult (10 week old) mouse (n=1) with a dose of 4×10¹¹ total vg. Spine and brain of the mouse was harvested two weeks post injection for NGS analysis. As shown in FIG. 10, AAV2-M1 and AAV2-L5 produced elevated transgene expression in the brain, cervical spine and lumbar spine compared to AAV2, AAV8 and AAV9.

The studies described herein produced previously-undescribed AAV capsids that, when vectorised, had the ability to transduce cells of the human CNS, including cells in the retina and/or brain, indicating that such AAV may have particular utility in gene therapy applications to treat diseases and conditions associated with the CNS, and in particular diseases and conditions of the human retina or brain. As the retinal explant culture system was designed to simulate intravitreal delivery, the effectiveness of the AAV variants to transduce the cells may be especially relevant for retinal gene therapy applications where intravitreal delivery may be beneficial, such as in instances where it is desirable to avoid complications associated with subretinal delivery, including subretinal detachment. This can have significant commercial applications, due to lower surgical risk with the therapy and the ability of intravitreal delivery of gene therapy to be performed as an outpatient procedure.

TABLE 4
Capsid Sequences

| SEQ | | |
|---|---|---|
| ID | Capsid | Capsid sequence |
| NO | name | (sequence corresponding to peptide insert in bold) |
| 1 | AAV2 | MAADGYLPDWLEDTLSEGIRQWWKLKPGPPPPKPAERHKDDSRGLVLPGYKYLGPFNGLD |
| | prototypic | KGEPVNEADAAALEHDKAYDRQLDSGDNPYLKYNHADAEFQERLKEDTSFGGNLGRAVFQ |
| | capsid -VP1 | AKKRVLEPLGLVEEPVKTAPGKKRPVEHSPVEPDSSSGTGKAGQQPARKRLNFGQTGDAD |
| | (protein) | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQRGNRQAATADVNTQGVLPG |
| | | MVWQDRDVYLQGPIWAKIPHTDGHFHPSPLMGGFGLKHPPPQILIKNTPVPANPSTTFSAA |
| | | KFASFITQYSTGQVSVEIEWELQKENSKRWNPEIQYTSNYNKSVNVDFTVDTNGVYSEPRP |
| | | IGTRYLTRNL |
| 2 | AAV2 1.1 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSAPPRHPSEAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL |
| 3 | AAV2 1.2 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRTTQFHPPEAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL |
| 4 | AAV2 1.3 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRPKQPTQPKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 5 | AAV2 1.4 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRPSYSPSNQAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 6 | AAV2 1.5 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSSVVSSRCEAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 7 | AAV2 L1 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRKNKDTPVKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 8 | AAV2 L2 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRNQNETKRQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL* |
| 9 | AAV2 L3 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSKQLPTNNKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 10 | AAV2 L4 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRQNPKLGSEAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 11 | AAV2 L5 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSKINPNASKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 12 | AAV2 L6 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSTRNPARNQAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 13 | AAV2 L7 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRSMGRGLGEAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 14 | AAV2 M1 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRRQRIPGGEAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 15 | AAV2 M2 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSNHHTNNPKAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL* |
| 16 | AAV2 M3 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRNYGRQDSQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL* |
| 17 | AAV2 M4 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSLPRRDAPKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 18 | AAV2 M5 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSLDKKNATKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 19 | AAV2 M6 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSSQRLPTTQAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 20 | AAV2 M7 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRPTKHLDREAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 21 | AAV2 H1 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRQQPQNTRQAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL* |
| 22 | AAV2 H2 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSTLQRTMAKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 23 | AAV2 H3 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRHLAVAPPQAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 24 | AAV2 H4 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRPWRESSQEAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL* |
| 25 | AAV2 H5 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQSTTTRDMPKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 26 | AAV2 H6 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRRKAENQMKAAATAD |
| | | VNTQGVLPGMVWQDRDVYLQGPIWAKIPHTDGHFHPSPLMGGFGLKHPPPQILIKNTPVPA |
| | | NPSTTFSAAKFASFITQYSTGQVSVEIEWELQKENSKRWNPEIQYTSNYNKSVNVDFTVDT |
| | | NGVYSEPRPIGTRYLTRNL* |
| 27 | AAV2 H7 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRRGSDPVRKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 28 | AAV2 H9 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRKKNEETKKAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 29 | AAV2 H11 | MAADGYLPDWLEDTLSEGIRQWWKLKPGPPPPKPAERHKDDSRGLVLPGYKYLGPFNGLD |
| | (protein) | KGEPVNEADAAALEHDKAYDRQLDSGDNPYLKYNHADAEFQERLKEDTSFGGNLGRAVFQ |
| | | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQGQRRINMATGQAAATADV |
| | | NTQGVLPGMVWQDRDVYLQGPIWAKIPHTDGHFHPSPLMGGFGLKHPPPQILIKNTPVPAN |
| | | PSTTFSAAKFASFITQYSTGQVSVEIEWELQKENSKRWNPEIQYTSNYNKSVNVDFTVDTN |
| | | GVYSEPRPIGTRYLTRNL* |
| 30 | AAV2 1.1 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTGCGCC |
| | | GCCTCGGCATCCGTCTGAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 31 | AAV2 1.2 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGACTAC |
| | | GCAGTTTCATCCGCCGGAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 32 | AAV2 1.3 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCCTAA |
| | | GCAGCCTACGCAGCCTAAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 33 | AAV2 1.4 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCCTTC |
| | | TTATAGTCCTTCGAATCAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 34 | AAV2 1.5 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTAGTGT |
| | | TGTGAGTTCGCGTTGTGAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 35 | AAV2 L1 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGAAGA |
| | | ATAAGGATACGCCGGTGAAGGCGGCCGCGACCGCAGATGTCAACACACAGGGAGTT |
| | | TTACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 36 | AAV2 L2 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGAATCA |
| | | GAATGAGACTAAGAGGCAGGCGGCCGCGACCGCAGATGTCAACACACAGGGAGTTT |
| | | TACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 37 | AAV2 L3 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTAAGCA |
| | | GCTTCCGACTAATAATAAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 38 | AAV2 L4 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCAGAA |
| | | TCCGAAGCTGGGTTCGGAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 39 | AAV2 L5 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTAAGAT |
| | | TAATCCGAATGCTTCGAAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 40 | AAV2 L6 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTACTCG |
| | | TAATCCGGCTAGGAATCAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 41 | AAV2 L7 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGAGTAT |
| | | GGGGCGGGGTCTGGGTGAGGCGGCCGCGACCGCAGATGTCAACACACAGGGAGTTT |
| | | TACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 42 | AAV2 M1 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCGGCA |
| | | GAGGATTCCGGGGGGGGAGGCGGCCGCGACCGCAGATGTCAACACACAGGGAGTTT |
| | | TACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 43 | AAV2 M2 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTAATCA |
| | | TCATACGAATAATCCGAAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 44 | AAV2 M3 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGAATTA |
| | | TGGTAGGCAGGATTCTCAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 45 | AAV2 M4 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TITTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTTTGCC |
| | | TCGGCGGGATGCTCCTAAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 46 | AAV2 M5 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTCTGGA |
| | | TAAGAAGAATGCGACGAAGGCGGCCGCGACCGCAGATGTCAACACACAGGGAGTTT |
| | | TACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 47 | AAV2 M6 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTAGTCA |
| | | GAGGCTGCCGACGACTCAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 48 | AAV2 M7 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCCGAC |
| | | GAAGCATTTGGATCGGGAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 49 | AAV2 H1 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCAGCA |
| | | GCCTCAGAATACTCGTCAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 50 | AAV2 H2 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTACGCT |
| | | TCAGCGGACGATGGCGAAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 51 | AAV2 H3 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCATCT |
| | | GGCTGTGGCGCCGCCGCAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 52 | AAV2 H4 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCCTTG |
| | | GCGTGAGTCGTCTCAGGAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 53 | AAV2 H5 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGTACGAC |
| | | TACGCGTGATATGCCTAAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 54 | AAV2 H6 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGAGGA |
| | | AGGCGGAGAATCAGATGAAGGCGGCCGCGACCGCAGATGTCAACACACAGGGAGTT |
| | | TTACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 55 | AAV2 H7 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGAGGG |
| | | GGAGTGATCCGGTTAGGAAGGCGGCCGCGACCGCAGATGTCAACACACAGGGAGTT |
| | | TTACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 56 | AAV2 H9 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TITTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGAAGA |
| | | AGAATGAGGAGACGAAGAAGGCGGCCGCGACCGCAGATGTCAACACACAGGGAGTT |
| | | TTACCTGGCATGGTGTGGCAAGACAGAGACGTGTACCTGCAGGGTCCCATTTGGGCCAA |
| | | GATTCCTCACACGGATGGACACTTTCACCCGTCTCCGCTGATGGGCGGCTTTGGACTGA |
| | | AACACCCGCCTCCTCAGATCCTCATCAAAAACACGCCTGTACCTGCGAATCCTTCGACCA |
| | | CCTTCAGCGCTGCAAAGTTTGCTTCATTCATCACCCAGTACTCCACTGGACAGGTCAGCG |
| | | TGGAGATTGAGTGGGAGCTGCAGAAGGAAAACAGCAAGCGCTGGAATCCCGAGATCCA |
| | | GTACACATCCAACTACAACAAATCTGTTAATGTGGACTTTACTGTGGACACTAATGGAGT |
| | | GTATAGTGAGCCTCGCCCCATTGGCACCCGTTACCTCACCCGTAATCTGTAA |
| 57 | AAV2 H11 | ATGGCTGCCGATGGTTATCTTCCAGATTGGCTCGAGGACACTCTCTCTGAAGGCATTCG |
| | (DNA) | CCAGTGGTGGAAGTTGAAACCTGGACCACCGCCACCCAAACCCGCAGAGCGGCATAAG |
| | | GACGACAGCCGGGGTCTTGTGCTTCCTGGTTACAAGTACCTCGGACCCTTCAACGGACT |
| | | CGACAAGGGGGAGCCCGTCAACGAGGCGGACGCAGCGGCCCTCGAGCACGACAAGGC |
| | | CTACGACCGGCAGCTCGACAGCGGTGACAACCCGTACCTCAAGTACAACCACGCCGAC |
| | | GCCGAGTTTCAGGAGCGTCTGAAAGAAGATACGTCTTTTGGGGGCAACCTCGGGCGAG |
| | | CAGTCTTCCAGGCCAAGAAGAGGGTTCTCGAACCTCTTGGTCTGGTTGAGGAACCTGTT |
| | | AAGACGGCTCCTGGAAAGAAGCGTCCGGTAGAGCACTCTCCAGTGGAGCCAGACTCCT |
| | | CCTCGGGCACCGGCAAGGCGGGCCAGCAGCCCGCTAGAAAGAGACTCAATTTTGGTCA |
| | | GACTGGCGACGCAGACTCAGTCCCCGACCCTCAACCTCTCGGACAGCCTCCAGCAGCCC |
| | | CCTCTGGTTTGGGAACTAATACAATGGCTACAGGCAGTGGCGCACCAATGGCAGACAAT |
| | | AACGAAGGCGCCGACGGAGTGGGTAATTCCTCGGGAAATTGGCATTGCGATTCCACATG |
| | | GATGGGCGACAGAGTCATCACCACCAGCACCCGAACCTGGGCCCTGCCCACCTACAACA |
| | | ACCACCTCTACAAGCAAATCAGTAGCCAATCAGGAGCCAGCAACGACAACCACTACTTC |
| | | GGCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCCAAGAGACTCAACTT |
| | | CAAGCTCTTCAACATCCAAGTCAAGGAGGTCACGCAGAATGATGGCACGACGACCATCG |
| | | CCAATAACCTTACCAGCACGGTTCAAGTGTTTACGGACTCGGAGTACCAGCTCCCGTAC |
| | | GTCCTCGGCTCTGCGCACCAGGGCTGCCTCCCTCCGTTCCCGGCGGACGTCTTCATGGT |
| | | GCCGCAGTACGGCTACCTGACGCTGAACAACGGCAGCCAGGCCGTGGGACGCTCATCC |
| | | TTTTACTGCCTGGAATACTTCCCTTCGCAGATGCTGAGAACGGGCAACAACTTTACCTTC |
| | | AGCTACACCTTTGAGGACGTGCCTTTCCACAGCAGCTACGCTCACAGCCAGAGCCTGGA |
| | | CCGGCTGATGAATCCTCTCATCGACCAGTACCTGTACTACCTGAGCAGAACTAACACTCC |
| | | ATCCGGAACCACCACGCAATCAAGGTTGCAGTTTAGCCAGGCCGGAGCGAGTGACATTC |
| | | GGGACCAGTCTAGAAACTGGCTACCTGGACCCTGTTACCGGCAGCAGCGCGTTTCAAAG |
| | | ACATCTGCGGACAACAACAACAGCGAATACTCGTGGACTGGTGCTACCAAATATCATCTC |
| | | AATGGCCGTGACTCTCTGGTGAATCCTGGCCCGGCTATGGCCTCACACAAAGACGACGA |
| | | AGAAAAGTTCTTTCCCCAGAGCGGTGTCCTCATTTTTGGCAAACAAGGCTCAGAGAAAAC |
| | | AAACGTGGATATTGAAAAGGTCATGATTACCGACGAAGAGGAAATTAGGACCACTAACC |
| | | CTGTGGCCACGGAGCAGTATGGGTCTGTGTCTACCAACCTCCAGGGCCAGAGGCGGAT |
| | | TAATATGGCGACGGGGCAGGCGGCCGCGACCGCAGATGTCAACACACAGGGAGTTTT |
| | | ACCTGGCATGGTGTGGCAAGACAGAGACGTGTACCTGCAGGGTCCCATTTGGGCCAAG |
| | | ATTCCTCACACGGATGGACACTTTCACCCGTCTCCGCTGATGGGCGGCTTTGGACTGAA |
| | | ACACCCGCCTCCTCAGATCCTCATCAAAAACACGCCTGTACCTGCGAATCCTTCGACCAC |
| | | CTTCAGCGCTGCAAAGTTTGCTTCATTCATCACCCAGTACTCCACTGGACAGGTCAGCGT |
| | | GGAGATTGAGTGGGAGCTGCAGAAGGAAAACAGCAAGCGCTGGAATCCCGAGATCCAG |
| | | TACACATCCAACTACAACAAATCTGTTAATGTGGACTTTACTGTGGACACTAATGGAGTG |
| | | TATAGTGAGCCTCGCCCCATTGGCACCCGTTACCTCACCCGTAATCTGTAA |
| 86 | AAV 7m8 | MAADGYLPDWLEDTLSEGIRQWWKLKPGPPPPKPAERHKDDSRGLVLPGYKYLGPFNGLD |
| | capsid (VP1 | KGEPVNEADAAALEHDKAYDRQLDSGDNPYLKYNHADAEFQERLKEDTSFGGNLGRAVFQ |
| | protein) | AKKRVLEPLGLVEEPVKTAPGKKRPVEHSPVEPDSSSGTGKAGQQPARKRLNFGQTGDAD |
| | | SVPDPQPLGQPPAAPSGLGTNTMATGSGAPMADNNEGADGVGNSSGNWHCDSTWMGD |
| | | RVITTSTRTWALPTYNNHLYKQISSQSGASNDNHYFGYSTPWGYFDFNRFHCHFSPRDWQ |
| | | RLINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFTFSYTFEDVPFH |
| | | SSYAHSQSLDRLMNPLIDQYLYYLSRTNTPSGTTTQSRLQFSQAGASDIRDQSRNWLPGPC |
| | | YRQQRVSKTSADNNNSEYSWTGATKYHLNGRDSLVNPGPAMASHKDDEEKFFPQSGVLIF |
| | | GKQGSEKTNVDIEKVMITDEEEIRTTNPVATEQYGSVSTNLQRGNLALGETTRPARQAATA |
| | | DVNTQGVLPGMVWQDRDVYLQGPIWAKIPHTDGHFHPSPLMGGFGLKHPPPQILIKNTPV |
| | | PANPSTTFSAAKFASFITQYSTGQVSVEIEWELQKENSKRWNPEIQYTSNYNKSVNVDFTV |
| | | DTNGVYSEPRPIGTRYLTRNL |
| 87 | AAV8 | MAADGYLPDWLEDNLSEGIREWWALKPGAPKPKANQQKQDDGRGLVLPGYKYLGPFNGL |
| | capsid (VP1 | DKGEPVNAADAAALEHDKAYDQQLQAGDNPYLRYNHADAEFQERLQEDTSFGGNLGRAVF |
| | protein) | QAKKRVLEPLGLVEEGAKTAPGKKRPVEPSPQRSPDSSTGIGKKGQQPARKRLNFGQTGDS |
| | | ESVPDPQPLGEPPAAPSGVGPNTMAAGGGAPMADNNEGADGVGSSSGNWHCDSTWLGD |
| | | RVITTSTRTWALPTYNNHLYKQISNGTSGGATNDNTYFGYSTPWGYFDFNRFHCHFSPRD |
| | | WQRLINNNWGFRPKRLSFKLFNIQVKEVTQNEGTKTIANNLTSTIQVFTDSEYQLPYVLGSA |
| | | HQGCLPPFPADVFMIPQYGYLTLNNGSQAVGRSSFYCLEYFPSQMLRTGNNFQFTYTFEDVP |
| | | FHSSYAHSQSLDRLMNPLIDQYLYYLSRTQTTGGTANTQTLGFSQGGPNTMANQAKNWLP |
| | | GPCYRQQRVSTTTGQNNNSNFAWTAGTKYHLNGRNSLANPGIAMATHKDDEERFFPSNGI |
| | | LIFGKQNAARDNADYSDVMLTSEEEIKTTNPVATEEYGIVADNLQQQNTAPQIGTVNSQGA |
| | | LPGMVWQNRDVYLQGPIWAKIPHTDGNFHPSPLMGGFGLKHPPPQILIKNTPVPADPPTTF |
| | | NQSKLNSFITQYSTGQVSVEIEWELQKENSKRWNPEIQYTSNYYKSTSVDFAVNTEGVYSE |
| | | PRPIGTRYLTRNL |
| 88 | AAV13 | MTDGYLPDWLEDNLSEGVREWWALQPGAPKPKANQQHQDNARGLVLPGYKYLGPGNGLD |
| | capsid (VP1 | KGEPVNAADAAALEHDKAYDQQLKAGDNPYLKYNHADAEFQERLQEDTSFGGNLGRAVFQ |
| | protein) | AKKRILEPLGLVEEAAKTAPGKKRPVEQSPAEPDSSSGIGKSGQQPARKRLNFGQTGDTES |
| | | VPDPQPLGQPPAAPSGVGSTTMASGGGAPMADNNEGADGVGNSSGNWHCDSQWLGDR |
| | | VITTSTRTWALPTYNNHLYKQISSQSGATNDNHYFGYSTPWGYFDFNRFHCHFSPRDWQR |
| | | LINNNWGFRPKRLNFKLFNIQVKEVTQNDGTTTIANNLTSTVQVFTDSEYQLPYVLGSAHQ |
| | | GCLPPFPADVFMVPQYGYLTLNNGSQAVGRSSFYCLEYFPSQMLRTGNNFQFSYTFEDVPF |
| | | HSSYAHSQSLDRLMNPLIDQYLYYLNRTQTASGTQQSRLLFSQAGPTSMSLQAKNWLPGP |
| | | CYRQQRLSKQANDNNNSNFPWTGATKYHLNGRDSLVNPGPAMASHKDDKEKFFPMHGTL |
| | | IFGKEGTNANNADLENVMITDEEEIRTTNPVATEQYGTVSNNLQNSNAGPTTGTVNHQGAL |
| | | PGMVWQDRDVYLQGPIWAKIPHTDGHFHPSPLMGGFGLKHPPPQIMIKNTPVPANPPTNFS |
| | | AAKFASFITQYSTGQVSVEIEWELQKENSKRWNPEIQYTSNYNKSVNVDFTVDTNGVYSEP |
| | | RPIGTRYLTRNL |
| 89 | AAV | MAADGYLPDWLEDNLSEGIREWWDLKPGAPKPKANQQKQDDGRGLVLPGYKYLGPFNGL |
| | Anc80L65 | DKGEPVNAADAAALEHDKAYDQQLKAGDNPYLRYNHADAEFQERLQEDTSFGGNLGRAVF |
| | (VP1 | QAKKRVLEPLGLVEEGAKTAPGKKRPVEQSPQEPDSSSGIGKKGQQPARKRLNFGQTGDS |
| | protein) | ESVPDPQPLGEPPAAPSGVGSNTMAAGGGAPMADNNEGADGVGNASGNWHCDSTWLGD |
| | | RVITTSTRTWALPTYNNHLYKQISSQSGGSTNDNTYFGYSTPWGYFDFNRFHCHFSPRDW |
| | | QRLINNNWGFRPKKLNFKLFNIQVKEVTTNDGTTTIANNLTSTVQVFTDSEYQLPYVLGSAH |
| | | QGCLPPFPADVFMIPQYGYLTLNNGSQAVGRSSFYCLEYFPSQMLRTGNNFQFSYTFEDVPF |
| | | HSSYAHSQSLDRLMNPLIDQYLYYLSRTQTTSGTAGNRTLQFSQAGPSSMANQAKNWLPG |
| | | PCYRQQRVSKTTNQNNNSNFAWTGATKYHLNGRDSLVNPGPAMATHKDDEDKFFPMSGV |
| | | LIFGKQGAGNSNVDLDNVMITNEEEIKTTNPVATEEYGTVATNLQSANTAPATGTVNSQGA |
| | | LPGMVWQDRDVYLQGPIWAKIPHTDGHFHPSPLMGGFGLKHPPPQILIKNTPVPANPPTTF |
| | | SPAKFASFITQYSTGQVSVEIEWELQKENSKRWNPEIQYTSNYNKSTNVDFAVDTNGVYSE |
| | | PRPIGTRYLTRNL |

1. An AAV capsid polypeptide, comprising a peptide modification relative to the AAV2 capsid polypeptide set forth in SEQ ID NO:1, wherein:
the peptide modification is in variable region 8 (VRVIII);
the peptide modification comprises a 7 amino insertion relative to the AAV2 capsid polypeptide set forth in SEQ ID NO:1, and comprises the sequence set forth in any one of SEQ ID Nos:58-85; and
the portion of the capsid polypeptide that is not the peptide modification comprises at least or about 80%, 81%, 82%, 83%, 84%, 85%, 86%, 87%, 88%, 89%, 90%, 91%, 92%, 93%, 94%, 95%, or 96% sequence identity to positions 1-735, 138-735 or 203-735 of SEQ ID NO:1.

2-30. (canceled)
